SEQUENCE LISTING (90 sequences)

SEQ ID NO: 1 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHNDWYFDLWGRGTLVTVSS

SEQ ID NO: 2 - protein, 5 aa, Artificial Sequence
  source: /note=“Description of Artificial Sequence Synthetic peptide”
IYPMF

SEQ ID NO: 3 - protein, 17 aa, Artificial Sequence
  source: /note=“Description of Artificial Sequence Synthetic peptide”
WIGPSGGITKYADSVKG

SEQ ID NO: 4 - protein, 10 aa, Artificial Sequence
  source: /note=“Description of Artificial Sequence Synthetic peptide”
EGHNDWYFDL

SEQ ID NO: 5 - protein, 118 aa, Artificial Sequence [sequence omitted]

SEQ ID NO: 6 - protein, 5 aa, Artificial Sequence
  source: /note=“Description of Artificial Sequence Synthetic peptide”
AYEMK

SEQ ID NO: 7 - protein, 17 aa, Artificial Sequence
  source: /note=“Description of Artificial Sequence Synthetic peptide”
VIGPSGGFTFYADSVKG

SEQ ID NO: 8 - protein, 9 aa, Artificial Sequence
  source: /note=“Description of Artificial Sequence Synthetic peptide”
EGDNDAFDI

SEQ ID NO: 9 - protein, 447 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSAYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 11 aa, Artificial Sequence
  source: /note=“Description of Artificial Sequence Synthetic peptide”
RASQDISRYLA

SEQ ID NO: 11 - protein, 7 aa, Artificial Sequence
  source: /note=“Description of Artificial Sequence Synthetic peptide”
DASNLQT

SEQ ID NO: 12 - protein, 9 aa, Artificial Sequence
  source: /note=“Description of Artificial Sequence Synthetic peptide”
QQYDTLHPS

SEQ ID NO: 13 - protein, 108 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIK

SEQ ID NO: 14 - protein, 11 aa, Artificial Sequence
  source: /note=“Description of Artificial Sequence Synthetic peptide”
RASQSVSSYLA

SEQ ID NO: 15 - protein, 7 aa, Artificial Sequence
  source: /note=“Description of Artificial Sequence Synthetic peptide”
DASNRAT

SEQ ID NO: 16 - protein, 10 aa, Artificial Sequence
  source: /note=“Description of Artificial Sequence Synthetic peptide”
QQRSNWPMYT

SEQ ID NO: 17 - protein, 215 aa, Artificial Sequence
  source: /note=“Description of Artificial Sequence Synthetic polypeptide”
DIQMTQSPATLSLSPGERATLSCRASQSVSSYLAWYQQKPGQAPRLLIYDASNRATGIPARFSGSGSGTDFTLTISSLEPEDFAVYYCQQRSNWPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 10 aa, Artificial Sequence
  source: /note=“Description of Artificial Sequence Synthetic peptide”
EGQYDWYFDV

SEQ ID NO: 19 - protein, 10 aa, Artificial Sequence
  source: /note=“Description of Artificial Sequence Synthetic peptide”
EGDYDWYFDL

SEQ ID NO: 20 - protein, 10 aa, Artificial Sequence
  source: /note=“Description of Artificial Sequence Synthetic peptide”
EGQYDWYFEL

SEQ ID NO: 21 - protein, 10 aa, Artificial Sequence
  source: /note=“Description of Artificial Sequence Synthetic peptide”
EADIDWFFDL

SEQ ID NO: 22 - protein, 10 aa, Artificial Sequence
  source: /note=“Description of Artificial Sequence Synthetic peptide”
EGHYDWYFDL

SEQ ID NO: 23 - protein, 10 aa, Artificial Sequence
  source: /note=“Description of Artificial Sequence Synthetic peptide”
EGRYDWYFDP

SEQ ID NO: 24 - protein, 10 aa, Artificial Sequence
  source: /note=“Description of Artificial Sequence Synthetic peptide”
EGDYDWYFGL

SEQ ID NO: 25 - protein, 10 aa, Artificial Sequence
  source: /note=“Description of Artificial Sequence Synthetic peptide”
EGRYDWYFDL

SEQ ID NO: 26 - protein, 10 aa, Artificial Sequence
  source: /note=“Description of Artificial Sequence Synthetic peptide”
ESHIDRYFDL

SEQ ID NO: 27 - protein, 10 aa, Artificial Sequence
  source: /note=“Description of Artificial Sequence Synthetic peptide”
EGQYDWYFDV

SEQ ID NO: 28 - protein, 10 aa, Artificial Sequence
  source: /note=“Description of Artificial Sequence Synthetic peptide”
EGHYNGYFDL

SEQ ID NO: 29 - protein, 10 aa, Artificial Sequence
  source: /note=“Description of Artificial Sequence Synthetic peptide”
EGYYDWYFDL

SEQ ID NO: 30 - protein, 10 aa, Artificial Sequence
  source: /note=“Description of Artificial Sequence Synthetic peptide”
EGTYDWYLDL

SEQ ID NO: 31 - protein, 10 aa, Artificial Sequence
  source: /note=“Description of Artificial Sequence Synthetic peptide”
EGYYDWYFEL

SEQ ID NO: 32 - protein, 10 aa, Artificial Sequence
  source: /note=“Description of Artificial Sequence Synthetic peptide”
EGLIDWFFDQ

SEQ ID NO: 33 - protein, 10 aa, Artificial Sequence
  source: /note=“Description of Artificial Sequence Synthetic peptide”
EGQFDWYFDL

SEQ ID NO: 34 - protein, 10 aa, Artificial Sequence
  source: /note=“Description of Artificial Sequence Synthetic peptide”
EGTYDWYFDL

SEQ ID NO: 35 - protein, 9 aa, Artificial Sequence
  source: /note=“Description of Artificial Sequence Synthetic peptide”
EGENDAFDV

SEQ ID NO: 36 - protein, 9 aa, Artificial Sequence
  source: /note=“Description of Artificial Sequence Synthetic peptide”
EGDNDAYDT

SEQ ID NO: 37 - protein, 9 aa, Artificial Sequence
  source: /note=“Description of Artificial Sequence Synthetic peptide”
EGTNDAFDI

SEQ ID NO: 38 - protein, 9 aa, Artificial Sequence
  source: /note=“Description of Artificial Sequence Synthetic peptide”
EGDNDAFDS

SEQ ID NO: 39 - protein, 9 aa, Artificial Sequence
  source: /note=“Description of Artificial Sequence Synthetic peptide”
EGDNDAFDT

SEQ ID NO: 40 - protein, 9 aa, Artificial Sequence
  source: /note=“Description of Artificial Sequence Synthetic peptide”
EGDNDAYDR

SEQ ID NO: 41 - protein, 9 aa, Artificial Sequence
  source: /note=“Description of Artificial Sequence Synthetic peptide”
EGDNDVFDS

SEQ ID NO: 42 - protein, 9 aa, Artificial Sequence
  source: /note=“Description of Artificial Sequence Synthetic peptide”
EGDDDVFDM

SEQ ID NO: 43 - protein, 9 aa, Artificial Sequence
  source: /note=“Description of Artificial Sequence Synthetic peptide”
EGYNDAFDF

SEQ ID NO: 44 - protein, 9 aa, Artificial Sequence
  source: /note=“Description of Artificial Sequence Synthetic peptide”
EGDDDAYDM

SEQ ID NO: 45 - protein, 9 aa, Artificial Sequence
  source: /note=“Description of Artificial Sequence Synthetic peptide”
EQDYDTYDL

SEQ ID NO: 46 - protein, 9 aa, Artificial Sequence
  source: /note=“Description of Artificial Sequence Synthetic peptide”
EGDDDAFDT

SEQ ID NO: 47 - protein, 9 aa, Artificial Sequence
  source: /note=“Description of Artificial Sequence Synthetic peptide”
EADDDAFDI

SEQ ID NO: 48 - protein, 9 aa, Artificial Sequence
  source: /note=“Description of Artificial Sequence Synthetic peptide”
EGENDAFDM

SEQ ID NO: 49 - protein, 9 aa, Artificial Sequence
  source: /note=“Description of Artificial Sequence Synthetic peptide”
EGEYDTYDI

SEQ ID NO: 50 - protein, 10 aa, Artificial Sequence
  source: /note=“Description of Artificial Sequence Synthetic peptide”
EGYYDWYFDQ

SEQ ID NO: 51 - protein, 107 aa, Artificial Sequence
  source: /note=“Description of Artificial Sequence Synthetic polypeptide”
DIQMTQSPSFLSASVGDSVAITCRASQDISRYLAWYQQRPGKAPKLLIYDASNLQTGVPSRFSGSGSGTDFTFTITSLQPEDFGTYYCQQYDTLHPSFGPGTTVDIK

SEQ ID NO: 52 - DNA, 1845 bp, Homo sapiens
atgctggcggggggcgtgaggagcatgcccagccccctcctggcctgctggcagcccatcctcctgctggtgctgggctcagtgctgtcaggctcggccacgggctgcccgccccgctgcgagtgctccgcccaggaccgcgctgtgctgtgccaccgcaagcgctttgtggcagtccccgagggcatccccaccgagacgcgcctgctggacctaggcaagaaccgcatcaaaacgctcaaccaggacgagttcgccagcttcccgcacctggaggagctggagctcaacgagaacatcgtgagcgccgtggagcccggcgccttcaacaacctcttcaacctccggacgctgggtctccgcagcaaccgcctgaagctcatcccgctaggcgtcttcactggcctcagcaacctgaccaagctggacatcagcgagaacaagattgttatcctgctggactacatgtttcaggacctgtacaacctcaagtcactggaggttggcgacaatgacctcgtctacatctctcaccgcgccttcagcggcctcaacagcctggagcagctgacgctggagaaatgcaacctgacctccatccccaccgaggcgctgtcccacctgcacggcctcatcgtcctgaggctccggcacctcaacatcaatgccatccgggactactccttcaagaggctctaccgactcaaggtcttggagatctcccactggccctacttggacaccatgacacccaactgcctctacggcctcaacctgacgtccctgtccatcacacactgcaatctgaccgctgtgccctacctggccgtccgccacctagtctatctccgcttcctcaacctctcctacaaccccatcagcaccattgagggctccatgttgcatgagctgctccggctgcaggagatccagctggtgggcgggcagctggccgtggtggagccctatgccttccgcggcctcaactacctgcgcgtgctcaatgtctctggcaaccagctgaccacactggaggaatcagtcttccactcggtgggcaacctggagacactcatcctggactccaacccgctggcctgcgactgtcggctcctgtgggtgttccggcgccgctggcggctcaacttcaaccggcagcagcccacgtgcgccacgcccgagtttgtccagggcaaggagttcaaggacttccctgatgtgctactgcccaactacttcacctgccgccgcgcccgcatccgggaccgcaaggcccagcaggtgtttgtggacgagggccacacggtgcagtttgtgtgccgggccgatggcgacccgccgcccgccatcctctggctctcaccccgaaagcacctggtctcagccaagagcaatgggcggctcacagtcttccctgatggcacgctggaggtgcgctacgcccaggtacaggacaacggcacgtacctgtgcatcgcggccaacgcgggcggcaacgactccatgcccgcccacctgcatgtgcgcagctactcgcccgactggccccatcagcccaacaagaccttcgctttcatctccaaccagccgggcgagggagaggccaacagcacccgcgccactgtgcctttccccttcgacatcaagaccctcatcatcgccaccaccatgggcttcatctctttcctgggcgtcgtcctcttctgcctggtgctgctgtttctctggagccggggcaagggcaacacaaagcacaacatcgagatcgagtatgtgccccgaaagtcggacgcaggcatcagctccgccgacgcgccccgcaagttcaacatgaagatgatatga

SEQ ID NO: 53 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGYYDWYFDQWGRGTLVTVSS

SEQ ID NO: 54 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGQYDWYFDVWGRGTLVTVSS

SEQ ID NO: 55 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGDYDWYFDLWGRGTLVTVSS

SEQ ID NO: 56 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGQYDWYFELWGRGTLVTVSS

SEQ ID NO: 57 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREADIDWFFDLWGRGTLVTVSS

SEQ ID NO: 58 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHYDWYFDLWGRGTLVTVSS

SEQ ID NO: 59 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGRYDWYFDPWGRGTLVTVSS

SEQ ID NO: 60 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGDYDWYFGLWGRGTLVTVSS

SEQ ID NO: 61 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGRYDWYFDLWGRGTLVTVSS

SEQ ID NO: 62 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCARESHIDRYFDLWGRGTLVTVSS

SEQ ID NO: 63 - protein, 119 aa, Artificial Sequence; identical to SEQ ID NO: 54
  source: /note=“Description of Artificial Sequence Synthetic polypeptide”

SEQ ID NO: 64 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGHYNGYFDLWGRGTLVTVSS

SEQ ID NO: 65 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGYYDWYFDLWGRGTLVTVSS

SEQ ID NO: 66 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDWYLDLWGRGTLVTVSS

SEQ ID NO: 67 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGYYDWYFELWGRGTLVTVSS

SEQ ID NO: 68 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGLIDWFFDQWGRGTLVTVSS

SEQ ID NO: 69 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGQFDWYFDLWGRGTLVTVSS

SEQ ID NO: 70 - protein, 119 aa, Artificial Sequence
  source: /note=“Description of Artificial Sequence Synthetic polypeptide”
EVQLLESGGGLVQPGGSLRLSCAASGFTFSIYPMFWVRQAPGKGLEWVSWIGPSGGITKYADSVKGRFTISRDNSKNTLYLQMNSLRAEDTATYYCAREGTYDWYFDLWGRGTLVTVSS

SEQ ID NO: 71 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGENDAFDVWGQGTTVTVSS

SEQ ID NO: 72 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAYDTWGQGTTVTVSS

SEQ ID NO: 73 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGTNDAFDIWGQGTTVTVSS

SEQ ID NO: 74 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDSWGQGTTVTVSS

SEQ ID NO: 75 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAFDTWGQGTTVTVSS

SEQ ID NO: 76 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDAYDRWGQGTTVTVSS

SEQ ID NO: 77 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDNDVFDSWGQGTTVTVSS

SEQ ID NO: 78 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDDDVFDMWGQGTTVTVSS

SEQ ID NO: 79 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGYNDAFDFWGQGTTVTVSS

SEQ ID NO: 80 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDDDAYDMWGQGTTVTVSS

SEQ ID NO: 81 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEQDYDTYDLWGQGTTVTVSS

SEQ ID NO: 82 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGDDDAFDTWGQGTTVTVSS

SEQ ID NO: 83 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEADDDAFDIWGQGTTVTVSS

SEQ ID NO: 84 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGENDAFDMWGQGTTVTVSS

SEQ ID NO: 85 - protein, 118 aa, Artificial Sequence
  source: /note=“Description of Artificial Sequence Synthetic polypeptide”
EVQLLESGGGLVQPGGSLRLSCAASGFTFSAYEMKWVRQAPGKGLEWVSVIGPSGGFTFYADSVKGRFTISRDNSKNTLYLQMNSLRAEDTAVYYCATEGEYDTYDIWGQGTTVTVSS

SEQ ID NO: 86 - protein, 614 aa, Homo sapiens
MLAGGVRSMPSPLLACWQPILLLVLGSVLSGSATGCPPRCECSAQDRAVLCHRKRFVAVPEGIPTETRLLDLGKNRIKTLNQDEFASFPHLEELELNENIVSAVEPGAFNNLFNLRTLGLRSNRLKLIPLGVFTGLSNLTKLDISENKIVILLDYMFQDLYNLKSLEVGDNDLVYISHRAFSGLNSLEQLTLEKCNLTSIPTEALSHLHGLIVLRLRHLNINAIRDYSFKRLYRLKVLEISHWPYLDTMTPNCLYGLNLTSLSITHCNLTAVPYLAVRHLVYLRFLNLSYNPISTIEGSMLHELLRLQEIQLVGGQLAVVEPYAFRGLNYLRVLNVSGNQLTTLEESVFHSVGNLETLILDSNPLACDCRLLWVFRRRWRLNFNRQQPTCATPEFVQGKEFKDFPDVLLPNYFTCRRARIRDRKAQQVFVDEGHTVQFVCRADGDPPPAILWLSPRKHLVSAKSNGRLTVFPDGTLEVRYAQVQDNGTYLCIAANAGGNDSMPAHLHVRSYSPDWPHQPNKTFAFISNQPGEGEANSTRATVPFPFDIKTLIIATTMGFISFLGVVLFCLVLLFLWSRGKGNTKHNIEIEYVPRKSDAGISSADAPRKFNMKMI

SEQ ID NO: 87 - protein, 619 aa, Homo sapiens
MQVSKRMLAGGVRSMPSPLLACWQPILLLVLGSVLSGSATGCPPRCECSAQDRAVLCHRKRFVAVPEGIPTETRLLDLGKNRIKTLNQDEFASFPHLEELELNENIVSAVEPGAFNNLFNLRTLGLRSNRLKLIPLGVFTGLSNLTKLDISENKIVILLDYMFQDLYNLKSLEVGDNDLVYISHRAFSGLNSLEQLTLEKCNLTSIPTEALSHLHGLIVLRLRHLNINAIRDYSFKRLYRLKVLEISHWPYLDTMTPNCLYGLNLTSLSITHCNLTAVPYLAVRHLVYLRFLNLSYNPISTIEGSMLHELLRLQEIQLVGGQLAVVEPYAFRGLNYLRVLNVSGNQLTTLEESVFHSVGNLETLILDSNPLACDCRLLWVFRRRWRLNFNRQQPTCATPEFVQGKEFKDFPDVLLPNYFTCRRARIRDRKAQQVFVDEGHTVQFVCRADGDPPPAILWLSPRKHLVSAKSNGRLTVFPDGTLEVRYAQVQDNGTYLCIAANAGGNDSMPAHLHVRSYSPDWPHQPNKTFAFISNQPGEGEANSTRATVPFPFDIKTLIIATTMGFISFLGVVLFCLVLLFLWSRGKGNTKHNIEIEYVPRKSDAGISSADAPRKFNMKM

SEQ ID NO: 88 - protein, 22 aa, Artificial Sequence
  source: /note=“Description of Artificial Sequence Synthetic peptide”
MDMRVPAQLLGLLLLWFPGSRC

SEQ ID NO: 89 - protein, 22 aa, Artificial Sequence
  source: /note=“Description of Artificial Sequence Synthetic peptide”
MDMRVPAQLLGLLLLWLPGARC

SEQ ID NO: 90 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

(19) United States
(12) Patent Application Publication
(10) Pub. No.: US 2022/0144945 A1
(43) Pub. Date: May 12, 2022

(54) PHARMACEUTICAL COMPOSITIONS CONTAINING ANTI-LINGO-1 ANTIBODIES

(71) Applicant: Biogen MA Inc., Cambridge, MA (US)

(72) Inventors: Jason Edward Fernandez, Boston, MA (US); Shantanu Virendra Sule, Arlington, MA (US)

(21) Appl. No.: 17/437,634

(22) PCT Filed: Mar. 10, 2020
(86) PCT No.: PCT/US20/21842

§ 371 Date: Sep. 9, 2021

Related U.S. Application Data
(60) Provisional application No. 62/816,668, filed on Mar. 11, 2019.
(60) Provisional application No. 62/817,323, filed on Mar. 12, 2019.

(51) Int. Cl. C07K 16/28; A61K 47/18; A61K 47/26

(52) U.S. Cl. CPC C07K 16/2803; A61K 2039/505; A61K 47/26; A61K 47/183

(57) ABSTRACT

Pharmaceutical compositions containing anti-LINGO-1 antibodies or LINGO-1-binding fragments thereof are provided. These pharmaceutical compositions find use in the treatment of CNS demyelinating diseases, such as multiple sclerosis and optic neuritis (e.g., acute optic neuritis).

### CROSS-REFERENCE TO RELATED APPLICATIONS

This application claims priority to U.S. Provisional Application No. 62/816,668, filed Mar. 11, 2019, and U.S. Provisional Application No. 62/817,323, filed Mar. 12, 2019. The content of each of the foregoing applications are incorporated by reference herein in their entirety.

### FIELD

The present application relates generally to pharmaceutical compositions comprising anti-LINGO-1 antibodies and uses thereof.

### BACKGROUND

Multiple sclerosis (MS) is a chronic inflammatory and degenerative disease of the central nervous system (CNS). Although the etiology is uncertain, evidence suggests that MS is in part an autoimmune disease directed against protein components of myelin resulting in CNS demyelination and dysfunction.

MS primarily affects adults, with clinical onset typically occurring between 20 and 40 years of age, with higher predominance in women compared to men (Weinshenker et al., Brain. 1989; 112 (Pt 1): 133-46). The disease can take on 4 different clinical courses: relapsing-remitting, primary progressive, secondary progressive, and progressive-relapsing. At diagnosis, most adults with MS (about 85%) are found to have relapsing-remitting MS (RRMS), about 10% have primary progressive MS, and about 5% have progressive-relapsing MS.

In the relapsing-remitting form, relapses, which are caused by discrete foci of inflammation in the CNS, are typically followed by recovery. Symptoms of relapses include loss of vision or double vision, numbness or tingling sensation in the extremities, muscle weakness, slurred speech, difficulty with coordination, and bladder dysfunction. As lesions can occur throughout the CNS, the signs and symptoms of the disease will vary depending on the location of the lesion. However, as the disease progresses, in addition to inflammation, axonal loss and irreversible neurological deficits are accumulated. Relapse frequency, as well as the rate at which the disease progresses, is highly variable in the adult MS population. Clinically, this progressive loss of function manifests as progressive disability and over time, RRMS patients typically become secondary progressive MS (SPMS) patients in their disease course with an accompanying accumulation of physical disability and cognitive decline (Weinshenker et al., Brain. 1989; 112 (Pt 1): 133-46). As patients progress from RRMS to SPMS, they are more likely to experience progressive neurological decline with or without superimposed relapse.

The median time to progression from RRMS to SPMS is approximately 10 years (Runmarker and Andersen, Brain. 1993; 116(Pt 1):117-34). Approximately half of all MS patients are unable to walk without assistance within 15 years of their initial diagnosis (Runmarker and Andersen 1993; Weinshenker 1989), and more than half of patients die from MS or its complications (Bronnum-Hansen et al., Brain. 2004; 127(Pt 4):844-50).

Optic neuritis, e.g., acute optic neuritis (AON), is characterized by inflammatory white matter lesions in the optic nerve. It is often associated with MS and is one the most common initial manifestations of the disease. AON causes structural and functional optic nerve damage (e.g., neuroaxonal injury and demyelination) that can result in permanent visual impairment for some patients (Cole, S. R. et al. Invest Ophtalmol Vis Sci (2000) 41(5):1017-1021; Mi, S. et al. CNS Drugs 2013: 27(7):493-503; Mangione C M et al. Arch Ophthalmol. (1988) 116(11):1496-1504). The current treatment for acute optic neuritis is high dose steroids which provides mostly symptomatic relief and fails to enhance CNS remyelination or provide neuroaxonal protection (Beck R W et al. N Engl J Med 1992 326:581-8).

Currently approved therapies for MS are primarily immunomodulatory, and typically do not have direct effects on CNS repair. Although some degree of axonal remyelination by oligodendrocytes takes place early during the course of MS, typically, in younger patients, the ability to repair the CNS eventually fails, leading to irreversible tissue injury and an increase in disease-related disabilities. Thus, there is a need for additional therapies that enhance remyelination and neuroaxonal protection in CNS demyelinating diseases, such as MS and optic neuritis.

### SUMMARY

This disclosure relates, in part, to pharmaceutical compositions containing anti-LINGO-1 antibody or LINGO-1-binding fragments thereof and their use in the treatment of CNS demyelinating diseases, such as multiple sclerosis and optic neuritis.

In one aspect, the disclosure features a pharmaceutical composition comprising an anti-LINGO-1 antibody or LINGO-1-binding fragment thereof, arginine (e.g., the free-base form of arginine, such as L-arginine, or arginine hydrochloride, such as L-arginine hydrochloride, or a combination of free-base form arginine and arginine hydrochloride), and histidine (e.g., in the form of free-base form of histidine, such as L-histidine, or histidine hydrochloride, such as L-histidine hydrochloride, or a combination of free-base form histidine and histidine hydrochloride).

In some embodiments, the anti-LINGO-1 antibody or LINGO-1-binding fragment thereof comprises an immunoglobulin heavy chain variable domain (VH) and an immunoglobulin light chain variable domain (VL), the VH and VL comprising the CDRs of Li81. In some instances, the six CDRs of Li81 comprise or consist of the amino acid sequences set forth in SEQ ID NO:6 (VH-CDR1); SEQ ID NO:7 (VH-CDR2); SEQ ID NO:8 (VH-CDR3); SEQ ID NO:14 (VL-CDR1); SEQ ID NO:15 (VL-CDR2); and SEQ ID NO:16 (VL-CDR3).

In some embodiments, the invention provides a pharmaceutical composition comprising an anti-LINGO-1 antibody or LINGO-1-binding fragment thereof, histidine (e.g., in the form of free-base form of histidine, such as L-histidine, or histidine hydrochloride, such as L-histidine hydrochloride, or a combination of free-base form histidine and histidine hydrochloride), and at least one additional excipient selected from the group consisting of proline (e.g., in the form of free-base form of proline, such as L-proline, or proline hydrochloride, such as L-proline hydrochloride, or a combination of free-base form proline and proline hydrochloride) and methionine (e.g., e.g., in the form of free-base form of methionine, such as L-methionine, or methionine hydrochloride, such as L-methionine hydrochloride, or a combination of free-base form methionine and methionine hydrochloride), wherein the anti-LINGO-1 antibody or LINGO-1-binding fragment comprises an immunoglobulin heavy chain variable domain (VH) and an immunoglobulin light chain variable domain (VL), the VH and VL, respectively, comprising: (a) VH complementarity determining regions (CDRs), wherein VH-CDR1 comprises the amino acid sequence set forth in SEQ ID NO:6; VH-CDR2 comprises the amino acid sequence set forth in SEQ ID NO:7; and VH-CDR3 comprises the amino acid sequence set forth in SEQ ID NO:8; and (b) VL CDRs, wherein VL-CDR1 comprises the amino acid sequence set forth in SEQ ID NO:14; VL-CDR2 comprises the amino acid sequence set forth in SEQ ID NO:15; and VL-CDR3 comprises the amino acid sequence set forth in SEQ ID NO:16, and wherein the composition has a pH of about 6.0 to about 7.0. In some embodiments, the composition further comprises arginine hydrochloride (e.g., L-arginine hydrochloride).

In some embodiments, the composition comprises the anti-LINGO-1 antibody or LINGO-1-binding fragment thereof at a concentration of about 50 mg/ml to about 300 mg/ml. In some embodiments, the composition comprises the anti-LINGO-1 antibody or LINGO-1-binding fragment thereof at a concentration of about 100 mg/ml to about 250 mg/ml. In other embodiments, the composition comprises the anti-LINGO-1 antibody or LINGO-1-binding fragment thereof at a concentration of about 150 mg/ml to about 225 mg/ml. In other embodiments, the composition comprises the anti-LINGO-1 antibody or LINGO-1-binding fragment thereof at a concentration of about 175 mg/ml to about 220 mg/ml. In certain embodiments, the composition comprises the anti-LINGO-1 antibody or LINGO-1-binding fragment thereof at a concentration of about 200 mg/ml.

In some embodiments, the composition further comprises arginine (e.g., Arg HCl). In some embodiments, the composition further comprises arginine hydrochloride (e.g., L-arginine hydrochloride) at a concentration of about 50 mM to about 250 mM. In other embodiments, the composition comprises Arg HCl at a concentration of about 70 mM to about 170 mM. In other embodiments, the composition comprises Arg HCl at a concentration of about 75 mM to about 175 mM. In certain embodiments, the composition comprises Arg HCl at a concentration of about 80 mM. In certain embodiments, the composition comprises Arg HCl at a concentration of about 100 mM. In certain embodiments, the composition comprises Arg HCl at a concentration of about 120 mM. In certain embodiments, the composition comprises Arg HCl at a concentration of about 140 mM. In some embodiments, the composition comprises Arg HCl at a concentration of about 160 mM.

In some embodiments, the composition further comprises Polysorbate-80 (PS80). In some embodiments, the composition comprises PS80 at a concentration of about 0.01% to about 0.1%. In other embodiments, the composition comprises PS80 at a concentration of about 0.03% to about 0.08%. In certain embodiments, the composition comprises PS80 at a concentration of about 0.04%. In certain embodiments, the composition comprises PS80 at a concentration of about 0.05%. In certain embodiments, the composition comprises PS80 at a concentration of about 0.06%.

In some embodiments, the composition further comprises poloxamer 188. In some embodiments, the composition comprises poloxamer 188 at a concentration of about 0.01% to about 0.1%. In other embodiments, the composition comprises poloxamer 188 at a concentration of about 0.03% to about 0.08%. In certain embodiments, the composition comprises poloxamer 188 at a concentration of about 0.04%. In certain embodiments, the composition comprises poloxamer 188 at a concentration of about 0.05%. In certain embodiments, the composition comprises poloxamer 188 at a concentration of about 0.06%.

In some embodiments, the composition comprises histidine as an excipient. In certain embodiments, the composition comprises histidine (as free-base form of histidine, such as L-histidine, or histidine hydrochloride, such as L-histidine hydrochloride, or a combination of free-base form histidine and histidine hydrochloride) at a concentration of about 5 mM to about 30 mM. In certain embodiments, the composition comprises histidine (as free-base form of histidine, such as L-histidine, or histidine hydrochloride, such as L-histidine hydrochloride, or a combination of free-base form histidine and histidine hydrochloride) at a concentration of about 10 mM to about 30 mM. In certain embodiments, the composition comprises histidine (as free free-base form of histidine, such as L-histidine, or histidine hydrochloride, such as L-histidine hydrochloride, or a combination of free-base form histidine and histidine hydrochloride) at a concentration of about 15 mM to about 25 mM. In certain embodiments, the composition comprises histidine (as free-base form of histidine, such as L-histidine, or histidine hydrochloride, such as L-histidine hydrochloride, or a combination of free-base form histidine and histidine hydrochloride) at a concentration of about 20 mM.

In some embodiments, the composition further comprises methionine (as free-base form of methionine, such as L-methionine, or methionine hydrochloride, such as L-methionine hydrochloride, or a combination of free-base form methionine and methionine hydrochloride) as an excipient. In some embodiments, the methionine (as free-base form of methionine, such as L-methionine, or methionine hydrochloride, such as L-methionine hydrochloride, or a combination of free-base form methionine and methionine hydrochloride) at a concentration of about 5 mM to about 15 mM.

In some embodiments, the composition further comprises proline (as free-base form of proline, such as L-proline, or proline hydrochloride, such as L-proline hydrochloride, or a combination of free-base form proline and proline hydrochloride) as an excipient. In some embodiments, the composition comprises proline (as free-base form of proline, such as L-proline, or proline hydrochloride, such as L-proline hydrochloride, or a combination of free-base form proline and proline hydrochloride) at a concentration of about 140 mM to about 180 mM.

In some embodiments, the composition has a pH of about 5.8 to about 7.0. In certain embodiments, the composition has a pH of about 6.2 to about 6.8. In certain embodiments, the composition has a pH of about 6.2 to about 6.7. In some embodiments, the composition has a pH of about 6.3 to about 6.7. In other embodiments, the composition has a pH of about 6.4. In other embodiments, the composition has a pH of about 6.5.

In various embodiments, the pharmaceutical composition does not contain citrate.

In certain embodiments, the pharmaceutical composition comprises the anti-LINGO-1 antibody or LINGO-1-binding fragment at a concentration of about 150 mg/ml to about 300 mg/ml; arginine (e.g., the free-base form of arginine, such as L-arginine, or arginine hydrochloride, such as L-arginine hydrochloride, or a combination of free-base form arginine and arginine hydrochloride) at a concentration of about 70 mM to about 180 mM; histidine (e.g., in the form of free-base form of histidine, such as L-histidine, or histidine hydrochloride, such as L-histidine hydrochloride, or a combination of free-base form histidine and histidine hydrochloride) at a concentration of about 5 mM to about 30 mM; methionine (e.g., in the form of free-base form of methionine, such as L-methionine, or methionine hydrochloride, such as L-methionine hydrochloride, or a combination of free-base form methionine and methionine hydrochloride) at a concentration of about 5 mM to about 15 mM; and polysorbate 80 at a concentration of about 0.02% to about 0.08%. In some cases, this composition has a pH of about 5.8 to about 7.0. In some cases, this composition has a pH of about 6.2 to about 6.8. In certain embodiments, the composition has a pH of about 6.3 to about 6.7. In other embodiments, the composition has a pH of about 6.5.

In certain embodiments, the pharmaceutical composition comprises the anti-LINGO-1 antibody or LINGO-1-binding fragment at a concentration of about 150 mg/ml to about 300 mg/ml; arginine (e.g., the free-base form of arginine, such as L-arginine, or arginine hydrochloride, such as L-arginine hydrochloride, or a combination of free-base form arginine and arginine hydrochloride) at a concentration of about 70 mM to about 180 mM; histidine (e.g., in the form of free-base form of histidine, such as L-histidine, or histidine hydrochloride, such as L-histidine hydrochloride, or a combination of free-base form histidine and histidine hydrochloride) at a concentration of about 5 mM to about 30 mM; proline (e.g., in the form of free-base form of proline, such as L-proline, or proline hydrochloride, such as L-proline hydrochloride, or a combination of free-base form proline and proline hydrochloride) at a concentration of about 140 mM to about 180 mM; and polysorbate 80 at a concentration of about 0.01% to about 0.1% (e.g., of about 0.04% to about 0.06%). In some cases, this composition has a pH of about 5.8 to about 6.8. In some cases, this composition has a pH of about 6.0 to about 6.8. In certain embodiments, the composition has a pH of about 6.3 to about 6.7. In other embodiments, the composition has a pH of about 6.5.

In certain embodiments, the pharmaceutical composition comprises the anti-LINGO-1 antibody or LINGO-1-binding fragment at a concentration of about 175 mg/ml to about 225 mg/ml; arginine (e.g., the free-base form of arginine, such as L-arginine, or arginine hydrochloride, such as L-arginine hydrochloride, or a combination of free-base form arginine and arginine hydrochloride) at a concentration of about 150 mM to about 175 mM; histidine (e.g., in the form of free-base form of histidine, such as L-histidine, or histidine hydrochloride, such as L-histidine hydrochloride, or a combination of free-base form histidine and histidine hydrochloride) at a concentration of about 15 mM to about 25 mM; methionine (e.g., in the form of free-base form of methionine, such as L-methionine, or methionine hydrochloride, such as L-methionine hydrochloride, or a combination of free-base form methionine and methionine hydrochloride) at a concentration of about 5 mM to about 15 mM and polysorbate 80 at a concentration of about 0.04% to about 0.06%. In some cases, this composition has a pH of about 5.8 to about 6.8. In some cases, this composition has a pH of about 6.0 to about 6.8. In certain embodiments, the composition has a pH of about 6.3 to about 6.7. In other embodiments, the composition has a pH of about 6.5.

In certain embodiments, the pharmaceutical composition comprises the anti-LINGO-1 antibody or LINGO-1-binding fragment at a concentration of about 175 mg/ml to about 225 mg/ml; arginine (e.g., the free-base form of arginine, such as L-arginine, or arginine hydrochloride, such as L-arginine hydrochloride, or a combination of free-base form arginine and arginine hydrochloride) at a concentration of about 150 mM to about 175 mM; histidine (e.g., in the form of free-base form of histidine, such as L-histidine, or histidine hydrochloride, such as L-histidine hydrochloride, or a combination of free-base form histidine and histidine hydrochloride) at a concentration of about 15 mM to about 25 mM; methionine (e.g., in the form of free-base form of methionine, such as L-methionine, or methionine hydrochloride, such as L-methionine hydrochloride, or a combination of free-base form methionine and methionine hydrochloride) at a concentration of about 5 mM to about 15 mM; and poloxamer 188 at a concentration of about 0.04% to about 0.06%. In some cases, this composition has a pH of about 5.8 to about 6.8. In some cases, this composition has a pH of about 6.0 to about 6.8. In certain embodiments, the composition has a pH of about 6.3 to about 6.7. In other embodiments, the composition has a pH of about 6.5.

In certain embodiments, the pharmaceutical composition comprises the anti-LINGO-1 antibody or LINGO-1-binding fragment at a concentration of about 175 mg/ml to about 225 mg/ml; arginine hydrochloride (e.g., L-arginine hydrochloride) at a concentration of about 70 mM to about 90 mM; histidine (e.g., in the form of free-base form of histidine, such as L-histidine, or histidine hydrochloride, such as L-histidine hydrochloride, or a combination of free-base form histidine and histidine hydrochloride) at a concentration of about 15 mM to about 25 mM; proline (e.g., in the form of free-base form of proline, such as L-proline, or proline hydrochloride, such as L-proline hydrochloride, or a combination of free-base form proline and proline hydrochloride) at a concentration of about 140 mM to about 180 mM; and polysorbate 80 at a concentration of about 0.04% to about 0.06%. In some cases, this composition has a pH of about 5.8 to about 6.8. In some cases, this composition has a pH of about 6.0 to about 6.8. In certain embodiments, the composition has a pH of about 6.3 to about 6.7. In other embodiments, the composition has a pH of about 6.5.

In certain embodiments, the pharmaceutical composition comprises the anti-LINGO-1 antibody at a concentration of about 175 mg/ml to about 225 mg/ml, wherein the VH comprises the amino acid sequence set forth in SEQ ID NO:5 and the VL comprises the amino acid sequence set forth in SEQ ID NO:13; L-arginine hydrochloride at a concentration of about 150 mM to about 175 mM; L-histidine at a concentration of about 10 mM to about 30 mM; L-methionine at a concentration of about 5 mM to about 15 mM; and polysorbate-80 at a concentration of about 0.01% to about 0.1%; and the pharmaceutical composition has a pH of about 6.2 to 6.8.

In certain embodiments, the pharmaceutical composition comprises the anti-LINGO-1 antibody at a concentration of 175 mg/ml to 225 mg/ml, wherein the VH comprises the amino acid sequence set forth in SEQ ID NO:5 and the VL comprises the amino acid sequence set forth in SEQ ID NO:13; L-arginine hydrochloride at a concentration of 150 mM to 175 mM; L-histidine at a concentration of 10 mM to 30 mM; L-methionine at a concentration of 5 mM to 15 mM; and polysorbate-80 at a concentration of 0.01% to 0.1%; and the pharmaceutical composition has a pH of 6.2 to 6.8.

In certain embodiments, the pharmaceutical composition comprises the anti-LINGO-1 antibody at a concentration of about 175 mg/ml to about 225 mg/ml, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17; L-arginine hydrochloride at a concentration of about 150 mM to about 175 mM; L-histidine at a concentration of about 10 mM to about 30 mM; L-methionine at a concentration of about 5 mM to about 15 mM; and polysorbate-80 at a concentration of about 0.01% to about 0.1%; and the pharmaceutical composition has a pH of about 6.2 to 6.8.

In certain embodiments, the pharmaceutical composition comprises the anti-LINGO-1 antibody at a concentration of 175 mg/ml to 225 mg/ml, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17; L-arginine hydrochloride at a concentration of 150 mM to 175 mM; L-histidine at a concentration of 10 mM to 30 mM; L-methionine at a concentration of 5 mM to 15 mM; and polysorbate-80 at a concentration of 0.01% to 0.1%; and the pharmaceutical composition has a pH of 6.2 to 6.8.

In certain embodiments, the pharmaceutical composition comprises the anti-LINGO-1 antibody at a concentration of about 200 mg/ml, wherein the VH comprises the amino acid sequence set forth in SEQ ID NO:5 and the VL comprises the amino acid sequence set forth in SEQ ID NO:13; L-arginine hydrochloride at a concentration of about 160 mM; L-histidine at a concentration of about 20 mM; L-methionine at a concentration of about 10 mM; and polysorbate-80 at a concentration of about 0.05%; and the pharmaceutical composition has a pH of about 6.5.

In certain embodiments, the pharmaceutical composition comprises the anti-LINGO-1 antibody at a concentration of 200 mg/ml, wherein the VH comprises the amino acid sequence set forth in SEQ ID NO:5 and the VL comprises the amino acid sequence set forth in SEQ ID NO:13; L-arginine hydrochloride at a concentration of 160 mM; L-histidine at a concentration of 20 mM; L-methionine at a concentration of 10 mM; and polysorbate-80 at a concentration of 0.05%; and the pharmaceutical composition has a pH of 6.5.

In certain embodiments, the pharmaceutical composition comprises the anti-LINGO-1 antibody at a concentration of about 200 mg/ml, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17; L-arginine hydrochloride at a concentration of about 160 mM; L-histidine at a concentration of about 20 mM; L-methionine at a concentration of about 10 mM; and polysorbate-80 at a concentration of about 0.05%; and the pharmaceutical composition has a pH of about 6.5.

In certain embodiments, the pharmaceutical composition comprises the anti-LINGO-1 antibody at a concentration of 200 mg/ml, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17; L-arginine hydrochloride at a concentration of 160 mM; L-histidine at a concentration of 20 mM; L-methionine at a concentration of 10 mM; and polysorbate-80 at a concentration of 0.05%; and the pharmaceutical composition has a pH of 6.5.

In certain embodiments, the pharmaceutical composition comprises the anti-LINGO-1 antibody at a concentration of about 175 mg/ml to about 225 mg/ml, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17; L-arginine hydrochloride at a concentration of about 150 mM to about 175 mM; L-histidine at a concentration of about 10 mM to about 30 mM; L-methionine at a concentration of about 5 mM to about 15 mM; and poloxamer 188 at a concentration of about 0.01% to about 0.1%; and the pharmaceutical composition has a pH of about 6.2 to 6.8.

In certain embodiments, the pharmaceutical composition comprises the anti-LINGO-1 antibody at a concentration of 175 mg/ml to 225 mg/ml, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17; L-arginine hydrochloride at a concentration of 150 mM to 175 mM; L-histidine at a concentration of 10 mM to 30 mM; L-methionine at a concentration of 5 mM to 15 mM; and poloxamer 188 at a concentration of 0.01% to 0.1%; and the pharmaceutical composition has a pH of 6.2 to 6.8.

In certain embodiments, the pharmaceutical composition comprises the anti-LINGO-1 antibody at a concentration of about 200 mg/ml, wherein the VH comprises the amino acid sequence set forth in SEQ ID NO:5 and the VL comprises the amino acid sequence set forth in SEQ ID NO:13; L-arginine hydrochloride at a concentration of about 160 mM; L-histidine at a concentration of about 20 mM; L-methionine at a concentration of about 10 mM; and poloxamer 188 at a concentration of about 0.05%; and the pharmaceutical composition has a pH of about 6.5.

In certain embodiments, the pharmaceutical composition comprises the anti-LINGO-1 antibody at a concentration of 200 mg/ml, wherein the VH comprises the amino acid sequence set forth in SEQ ID NO:5 and the VL comprises the amino acid sequence set forth in SEQ ID NO:13; L-arginine hydrochloride at a concentration of 160 mM; L-histidine at a concentration of 20 mM; L-methionine at a concentration of 10 mM; and poloxamer 188 at a concentration of 0.05%; and the pharmaceutical composition has a pH of 6.5.

In certain embodiments, the pharmaceutical composition comprises the anti-LINGO-1 antibody at a concentration of about 200 mg/ml, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17; L-arginine hydrochloride at a concentration of about 160 mM; L-histidine at a concentration of about 20 mM; L-methionine at a concentration of about 10 mM; and poloxamer 188 at a concentration of about 0.05%; and the pharmaceutical composition has a pH of about 6.5.

In certain embodiments, the pharmaceutical composition comprises the anti-LINGO-1 antibody at a concentration of 200 mg/ml, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17; L-arginine hydrochloride at a concentration of 160 mM; L-histidine at a concentration of 20 mM; L-methionine at a concentration of 10 mM; and poloxamer 188 at a concentration of 0.05%; and the pharmaceutical composition has a pH of 6.5.

In some embodiments, the pharmaceutical composition comprises an anti-LINGO-1 antibody or LINGO-1-binding fragment comprising a VH and a VL, wherein the VH comprises or consists of a sequence at least 80% identical to SEQ ID NO:5 and the VL comprises or consists of a sequence at least 80% identical to SEQ ID NO:13. In some embodiments, the VH comprises or consists of a sequence at least 90% identical to SEQ ID NO:5 and the VL comprises or consists of a sequence at least 90% identical to SEQ ID NO:13. In some embodiments, the VH comprises or consists of the sequence of SEQ ID NO:5 and the VL comprises or consists of the sequence of SEQ ID NO:13.

In some embodiments, the anti-LINGO-1 antibody comprises an immunoglobulin heavy chain and an immunoglobulin light chain. In certain instances, the heavy chain comprises or consists of a sequence at least 80% identical to SEQ ID NO:9 and the light chain comprises or consists of a sequence at least 80% identical to SEQ ID NO:17. In other instances, the heavy chain comprises or consists of a sequence at least 90% identical to SEQ ID NO:9 and the light chain comprises or consists of a sequence at least 90% identical to SEQ ID NO:17. In yet other instances, the heavy chain comprises or consists of the sequence of SEQ ID NO:9 and the light chain comprises or consists of the sequence of SEQ ID NO:17.

In certain embodiments, the pharmaceutical composition comprises a fixed dose of about 210 mg, about 225 mg, about 250 mg, about 350 mg, about 375 mg, about 750 mg, about 1050 mg, about 1125 mg, about 1250 mg, about 3150 mg, about 3375 mg, about 3500 mg, about 6300 mg, or about 6750 mg of the anti-LINGO-1 antibody or LINGO-1-binding fragment.

In another aspect, the disclosure features a method of treating a central nervous system (CNS) demyelinating disease in a human subject in need thereof. Non-limiting examples of CNS demyelinating diseases are multiple sclerosis and optic neuritis. The method comprises administering to the human subject a pharmaceutical composition described herein.

In some instances, the patient is currently, has previously, and/or in the future will be treated with an immunomodulatory agent including, without limitation, an inhibitor of dihydroorotate dehydrogenase (e.g., teriflunomide), interferon beta 1a, interferon beta 1b, glatiramer acetate, fingolimod, alemtuzumab, cladribine, ocrelizumab, peginterferon beta 1a, fumarate (e.g., dimethyl fumarate, diroximel fumarate, or monomethyl fumarate), an antibody to the alpha subunit of the interleukin 2 receptor, and/or natalizumab.

In some embodiments, of the above aspects, the pharmaceutical composition is administered subcutaneously to the human subject. In some embodiments, of these aspects, the pharmaceutical composition is administered intravenously to the human subject. In some embodiments, of these aspects, the pharmaceutical composition is administered intramuscularly to the human subject.

In certain instances in the above aspects, the anti-LINGO-1 antibody or LINGO-1-binding fragment at a dose of about 3 mg per kg, about 5 mg per kg, about 10 mg per kg, about 15 mg per kg, about 30 mg per kg, about 45 mg per kg, about 90 mg per kg, about 100 mg per kg, or about 120 mg per kg of body weight of the human subject.

In other instances in the above aspects, the pharmaceutical composition comprises a fixed dose of about 210 mg, about 225 mg, about 250 mg, about 350 mg, about 375 mg, about 750 mg, about 1125 mg, about 1250 mg, about 3150 mg, about 3375 mg, about 3500 mg, about 6300 mg, or about 6750 mg of the anti-LINGO-1 antibody or LINGO-1-binding fragment.

In yet another aspect, the disclosure features a syringe comprising a pharmaceutical composition described herein.

In yet additional aspects, the invention provides a kit comprising the syringe comprising a pharmaceutical composition described herein and an immunomodulatory agent (e.g., interferon beta 1a, interferon beta 1b, glatiramer acetate, fingolimod, alemtuzumab, cladribine, ocrelizumab, peginterferon beta 1a, fumarate (e.g., dimethyl fumarate, diroximel fumarate, or monomethyl fumarate), natalizumab, an antibody to the alpha subunit of the interleukin 2 receptor, an inhibitor of dihydroorotate dehydrogenase, a steroid, and/or a combination of two or more of the forgoing).

In some embodiments, the invention provides a kit comprising one or more syringes comprising the pharmaceutical composition described herein, wherein said one or more syringes is adapted for subcutaneous administration. In some embodiments, the kit further comprises instructions for administering the composition subcutaneously.

In some embodiments, the invention provides a kit comprising one or more syringes comprising the pharmaceutical composition described herein, wherein said one or more syringes is adapted for intravenous administration. In some embodiments, the kit further comprises instructions for administering the composition intravenously including, for example, instructions for diluting the composition in a physiologically acceptable liquid such as 0.9% NaCl.

In some embodiments, the invention provides a kit comprising one or more syringes comprising the pharmaceutical composition described herein, wherein said one or more syringes is adapted for intramuscular administration. In some embodiments, the kit further comprises instructions for administering the composition intramuscularly.

By the term “about” is meant the stated value+/−5%. For example, “about 100 mg/ml” means 95 mg/ml to 105 mg/ml; and “about pH 6.0” means a pH of 5.7 to 6.3.

By the term “fixed dose” is meant a physically discrete unit that is suited as a unitary dosage for the subject(s) to be treated. That is, each unit contains a predetermined quantity of antibody calculated to achieve a desired therapeutic concentration or effect in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 is a line graph showing the benefit of histidine buffer at elevated protein concentrations. Lower percentage of high molecular weight (HMW) means increased stability.

FIG. 2 is a scatter plot graph showing that aggregation decreases as formulation pH increases.

FIG. 3 is a scatter plot graph showing that arginine HCl concentration in the formulation does not significantly impact anti-Lingo 1 aggregation.

FIG. 4 is a three-dimensional graph showing that formulation pH has a significant effect on anti-Lingo-1 solution viscosity.

FIG. 5 is a bar graph showing the differences in aggregation in 225 mg/mL anti-Lingo-1 antibody-containing formulations when different excipients are included in the formulation. Lower aggregation means increased stability, with aggregation below 3% being more desired.

FIG. 6 is a bar graph showing the different viscosities of 225 mg/mL anti-Lingo-1 antibody-containing formulations when different excipients are included in the formulation.

FIG. 7 is a line graph showing the change in aggregate over time at 5° C., from long term developmental stability evaluation. At 24 months, the top line is Formulation 6, the middle line is Formulation 5, and the bottom line is Formulation 2.

FIG. 8 is a line graph showing the change in aggregate over time at 25° C., from long term developmental stability evaluation. At 12 months, the top line is Formulation 1, the next line below is Formulation 5, the next line below is Formulation 3, the next line below is Formulation 6, and the bottom line is Formulation 2.

FIG. 9 is a line graph showing long-term aggregation stability data at 2-8° C., demonstrating the stability of the 200 mg/mL anti-Lingo-1 antibody, 20 mM histidine, 160 mM arginine HCl, 10 mM methionine, 0.05% polysorbate 80, pH 6.5 formulation.

### DETAILED DESCRIPTION

This application provides pharmaceutical compositions containing anti-LINGO-1 antibodies and LINGO-1-binding fragments thereof and their use in the treatment of CNS demyelinating diseases, such as MS and optic neuritis.

### LINGO-1

Leucine-rich repeat and immunoglobulin domain-containing Nogo receptor-interacting protein-1 (LINGO-1), previously called Sp35, is a cell surface glycoprotein that is selectively expressed in the adult CNS in neurons and oligodendrocytes, in the central nervous system (CNS) oligodendrocytes and neurons during development in normal circumstances, and is upregulated in CNS diseases. It contains a large extracellular domain with 12 leucine-rich repeat (LRR) motifs flanked by N- and C-terminal capping modules, one Ig domain of the I1 subtype, and a stalk region that is attached to a transmembrane region and a short cytoplasmic tail (Mi et al., 2004; Mosyak et al., J Biol Chem 281: 36378-36390, 2006). LINGO-1 suppresses oligodendrocyte differentiation, thereby preventing axonal myelination. Blocking its function leads to robust myelination in vitro and in animal models of demyelination.

LINGO-1 is a member of a protein family comprising 3 other paralogs: LINGO-2 (GI: 12309630, 61% protein identity), LINGO-3 (GI: 23342615, 56% identity) and LINGO-4 (GI: 21211752, 44% identity). LINGO-1 is highly conserved evolutionarily with human and mouse orthologues sharing 99.5% identity, and human and rat LINGO-1 sharing 99.5% identity. By Northern blot analysis, LINGO-1 was found to be highly expressed in human brain and was not detectable in non-neural tissues (Barrette et al. (2007) Mol Cell Neurosci, 34: 519-38; Carim-Todd et al. (2003) Eur Journal Neurosci, 18: 3167-82; Llorens et al. (2008) Dev Neurobiol, 68: 521-41; Mi et al. (2004) Nat Neurosci, 7: 221-8; Okafuji et al. (2005) Gene Expr Patterns, 6: 57-62; Park et al. (2006) Neurosci Lett, 404: 61-6; Shao et al. (2005) Neuron, 45: 353-9). LINGO-1 is developmentally regulated with expression in newborn rats peaking on postnatal day 1 and decreasing thereafter into adulthood (Ji et al., Mol Cell Neurosci. 2006; 33(3):311-20; Mi et al. (2004) Nat Neurosci, 7: 221-8; Mi et al. CNS Drugs. 2013; 27(7):493-503). Downregulation of LINGO-1 expression is associated with the onset of normal CNS myelination in rodents. LINGO-1 expression levels have been shown to be upregulated across neuropathologies, such as in animal models of spinal cord injury (Ji et al., Mol Cell Neurosci. 2006; 33(3):311-20) and glaucoma (Fu et al., Invest Ophthalmol Vis Sci. 2008; 49(3):975-85), in Parkinson's disease (Inoue Proc Natl Acad Sci USA. 2007; 104(36): 14430-5), and in MS lesions.

LINGO-1 has also been described in detail in International Applications PCT/US2006/026271, filed Jul. 7, 2006, PCT/US2004/008323, filed Mar. 17, 2004, PCT/US2005/022881, filed Jun. 24, 2005; PCT/US2008/000316, filed Jan. 9, 2008, PCT/US2017/041757; and PCT/US2016/012619, each of which is incorporated by reference in its entirety herein.

LINGO-1 is selectively expressed in both oligodendrocyte precursor cells (OPCs) and neurons. LINGO-1 functions as a negative regulator of oligodendrocyte differentiation myelination, and remyelination; preventing myelination of axons by oligodendrocytes (Lee et al. (2007) J Neurosci, 27: 220-5; Mi et al. (2005) Nat Neurosci, 8: 745-51; Mi et al. (2008) Int Journal Biochem Cell Biol 40(10):1971-8; Mi et al. (2009) Ann Neurology, 65: 304-15). Axonal and neuronal expression of LINGO-1 increases after injury (Ji et al. (2006) Mol Cell Neurosci, 33: 311-20). LINGO-1 expression prevents myelination of axons by oligodendrocytes. Several preclinical studies have demonstrated the potential for LINGO-1 antagonism to enhance CNS remyelination and neuroaxonal protection in animal models of toxic (Cuprizone) (Mi et al. (2009) Ann Neurology, 65: 304-15), chemical injury (lysophosphatidylcholine [LPC]), and inflammatory (myelin oligodendrocyte glycoprotein-experimental autoimmune encephalomyelitis [MOG-EAE]) (Mi et al. (2007) Nat Med, 13: 1228-33) demyelination; and of toxic (1-methyl-4-phenyl-1,2,3,6-tetrahydropyridine [MPTP]) neuronal injury (Inoue et al. (2007) Proc Natl Acad Sci, 104: 14430-5), traumatic/hypertensive optic nerve injury (Fu et al. (2008) Invest Opthalmol Vis Sci, 49: 975-85) and spinal cord injury (Ji et al. (2006) Mol Cell Neurosci, 33: 311-20; Ji et al. (2008) Mol Cell Neurosci, 39: 258-67; Lv et al. (2010) Neuroimmunomodulat, 17: 270-8). Remyelination and neuroaxonal protection can be provided via blockade of signaling by myelin debris and/or sulfated proteoglycans on the NgR1 receptor complex in the CNS caused by the inhibition of LINGO-1 in axons and oligodendroyctes. This in turn may enhance remyelination via differentiation of oligodendrocyte precursor cells (OPCs) normally present in the brain of MS patients. Thus, antagonism of LINGO-1 can enhance myelination or re-myelination of axons, e.g., by oligodendrocytes, and enhance neuroaxonal protection in the CNS, and for example, in CNS demyelinating diseases such as multiple sclerosis (MS) and acute optic neuritis, leading to improved CNS repair.

LINGO-1 is also known in the art by the names LRRN6, LRRN6A, FLJ14594, LERN1, MGC17422 and UNQ201. The human, full-length wild-type LINGO-1 polypeptide contains an LRR domain consisting of 14 leucine-rich repeats (including N- and C-terminal caps), an Ig domain, a transmembrane region, and a cytoplasmic domain. The cytoplasmic domain contains a canonical tyrosine phosphorylation site. In addition, the naturally occurring LINGO-1 protein contains a signal sequence, a short basic region between the LRR-C-terminal domain (LRRCT) and Ig domain, and a transmembrane region between the Ig domain and the cytoplasmic domain. Table 1 lists the LINGO-1 domains and other regions, according to amino acid residue number, based on the LINGO-1 amino acid sequence presented herein as SEQ ID NO: 86. The LINGO-1 polypeptide is characterized in more detail in PCT Publication No. WO 2004/085648, which is incorporated herein by reference in its entirety.

TABLE 1
LINGO-1 Domains

| Domain or Region | Beginning Residue | Ending Residue |
|---|---|---|
| Signal Sequence | 1 | 33 or 35 |
| LRRNT | 34 or 36 | 64 |
| LRR | 66 | 89 |
| LRR | 90 | 113 |
| LRR | 114 | 137 |
| LRR | 138 | 161 |
| LRR | 162 | 185 |
| LRR | 186 | 209 |
| LRR | 210 | 233 |
| LRR | 234 | 257 |
| LRR | 258 | 281 |
| LRR | 282 | 305 |
| LRR | 306 | 329 |
| LRR | 330 | 353 |
| LRRCT | 363 | 414 or 416 |
| Basic | 415 or 417 | 424 |
| Ig | 419 | 493 |
| Connecting sequence | 494 | 551 |
| Transmembrane | 552 | 576 |
| Cytoplasmic | 577 | 614 |

Tissue distribution and developmental expression of LINGO-1 has been studied in humans and rats. LINGO-1 biology has been studied in an experimental animal (rat) model. Expression of rat LINGO-1 is localized to neurons and oligodendrocytes, as determined by northern blot and immuno-histochemical staining. Rat LINGO-1 mRNA expression level is regulated developmentally, peaking shortly after birth, i.e., ca. postnatal day one. In a rat spinal cord transection injury model, LINGO-1 is up-regulated at the injury site, as determined by RT-PCR. See Mi et al. Nature Neurosci. 7:221-228 (2004).

In the context of the amino acids comprising the various structural and functional domains of a LINGO-1 polypeptide, the term “about” includes the particularly recited value and values larger or smaller by several (e.g., 10, 9, 8, 7, 6, 5, 4, 3, 2, or 1) amino acids. Since the location of these domains as listed in Table 1 have been predicted by computer graphics, one of ordinary skill would appreciate that the amino acid residues constituting the domains may vary slightly (e.g., by about 1 to 15 residues) depending on the criteria used to define the domain.

Full-length, wild-type LINGO-1 binds to NgR1. See PCT Publication No. WO 2004/085648. LINGO-1 is expressed in oligodendrocytes and that the LINGO-1 protein is involved in the regulation of oligodendrocyte-mediated myelination of axons. See U.S Patent Publication No. 2006/0009388 A1, which is incorporated herein by reference in its entirety.

The nucleotide sequence for the full-length LINGO-1 molecule is as follows:

(SEQ ID NO: 52)
ATGCTGGCGGGGGGCGTGAGGAGCATGCCCAGCCCCCTCCTGGCCTGCTG
GCAGCCCATCCTCCTGCTGGTGCTGGGCTCAGTGCTGTCAGGCTCGGCCA
CGGGCTGCCCGCCCCGCTGCGAGTGCTCCGCCCAGGACCGCGCTGTGCTG
TGCCACCGCAAGCGCTTTGTGGCAGTCCCCGAGGGCATCCCCACCGAGAC
GCGCCTGCTGGACCTAGGCAAGAACCGCATCAAAACGCTCAACCAGGACG
AGTTCGCCAGCTTCCCGCACCTGGAGGAGCTGGAGCTCAACGAGAACATC
GTGAGCGCCGTGGAGCCCGGCGCCTTCAACAACCTCTTCAACCTCCGGAC
GCTGGGTCTCCGCAGCAACCGCCTGAAGCTCATCCCGCTAGGCGTCTTCA
CTGGCCTCAGCAACCTGACCAAGCTGGACATCAGCGAGAACAAGATTGTT
ATCCTGCTGGACTACATGTTTCAGGACCTGTACAACCTCAAGTCACTGGA
GGTTGGCGACAATGACCTCGTCTACATCTCTCACCGCGCCTTCAGCGGCC
TCAACAGCCTGGAGCAGCTGACGCTGGAGAAATGCAACCTGACCTCCATC
CCCACCGAGGCGCTGTCCCACCTGCACGGCCTCATCGTCCTGAGGCTCCG
GCACCTCAACATCAATGCCATCCGGGACTACTCCTTCAAGAGGCTCTACC
GACTCAAGGTCTTGGAGATCTCCCACTGGCCCTACTTGGACACCATGACA
CCCAACTGCCTCTACGGCCTCAACCTGACGTCCCTGTCCATCACACACTG
CAATCTGACCGCTGTGCCCTACCTGGCCGTCCGCCACCTAGTCTATCTCC
GCTTCCTCAACCTCTCCTACAACCCCATCAGCACCATTGAGGGCTCCATG
TTGCATGAGCTGCTCCGGCTGCAGGAGATCCAGCTGGTGGGCGGGCAGCT
GGCCGTGGTGGAGCCCTATGCCTTCCGCGGCCTCAACTACCTGCGCGTGC
TCAATGTCTCTGGCAACCAGCTGACCACACTGGAGGAATCAGTCTTCCAC
TCGGTGGGCAACCTGGAGACACTCATCCTGGACTCCAACCCGCTGGCCTG
CGACTGTCGGCTCCTGTGGGTGTTCCGGCGCCGCTGGCGGCTCAACTTCA
ACCGGCAGCAGCCCACGTGCGCCACGCCCGAGTTTGTCCAGGGCAAGGAG
TTCAAGGACTTCCCTGATGTGCTACTGCCCAACTACTTCACCTGCCGCCG
CGCCCGCATCCGGGACCGCAAGGCCCAGCAGGTGTTTGTGGACGAGGGCC
ACACGGTGCAGTTTGTGTGCCGGGCCGATGGCGACCCGCCGCCCGCCATC
CTCTGGCTCTCACCCCGAAAGCACCTGGTCTCAGCCAAGAGCAATGGGCG
GCTCACAGTCTTCCCTGATGGCACGCTGGAGGTGCGCTACGCCCAGGTAC
AGGACAACGGCACGTACCTGTGCATCGCGGCCAACGCGGGCGGCAACGAC
TCCATGCCCGCCCACCTGCATGTGCGCAGCTACTCGCCCGACTGGCCCCA
TCAGCCCAACAAGACCTTCGCTTTCATCTCCAACCAGCCGGGCGAGGGAG
AGGCCAACAGCACCCGCGCCACTGTGCCTTTCCCCTTCGACATCAAGACC
CTCATCATCGCCACCACCATGGGCTTCATCTCTTTCCTGGGCGTCGTCCT
CTTCTGCCTGGTGCTGCTGTTTCTCTGGAGCCGGGGCAAGGGCAACACAA
AGCACAACATCGAGATCGAGTATGTGCCCCGAAAGTCGGACGCAGGCATC
AGCTCCGCCGACGCGCCCCGCAAGTTCAACATGAAGATGATATGA

The polypeptide sequence for the full-length LINGO-1 polypeptide is as follows:

(SEQ ID NO: 86)
MLAGGVRSMPSPLLACWQPILLLVLGSVLSGSATGCPPRCECSAQDRAVL
CHRKRFVAVPEGIPTETRLLDLGKNRIKTLNQDEFASFPHLEELELNENI
VSAVEPGAFNNLFNLRTLGLRSNRLKLIPLGVFTGLSNLTKLDISENKIV
ILLDYMFQDLYNLKSLEVGDNDLVYISHRAFSGLNSLEQLTLEKCNLTSI
PTEALSHLHGLIVLRLRHLNINAIRDYSFKRLYRLKVLEISHWPYLDTMT
PNCLYGLNLTSLSITHCNLTAVPYLAVRHLVYLRFLNLSYNPISTIEGSM
LHELLRLQEIQLVGGQLAVVEPYAFRGLNYLRVLNVSGNQLTTLEESVFH
SVGNLETLILDSNPLACDCRLLWVFRRRWRLNFNRQQPTCATPEFVQGKE
FKDFPDVLLPNYFTCRRARIRDRKAQQVFVDEGHTVQFVCRADGDPPPAI
LWLSPRKHLVSAKSNGRLTVFPDGTLEVRYAQVQDNGTYLCIAANAGGND
SMPAHLHVRSYSPDWPHQPNKTFAFISNQPGEGEANSTRATVPFPFDIKT
LIIATTMGFISFLGVVLFCLVLLFLWSRGKGNTKHNIEIEYVPRKSDAGI
SSADAPRKFNMKMI

The human LINGO-1 gene contains alternative translation start codons, so that six additional amino acids of the protein. Thus, in a variant form, the sequence of human LINGO-1 is provided below.

(SEQ ID NO: 87)
MQVSKRMLAG GVRSMPSPLL ACWQPILLLV LGSVLSGSAT
GCPPRCECSA QDRAVLCHRK RFVAVPEGIP TETRLLDLGK
NRIKTLNQDE FASFPHLEEL ELNENIVSAV EPGAFNNLFN
LRTLGLRSNR LKLIPLGVFT GLSNLTKLDI SENKIVILLD
YMFQDLYNLK SLEVGDNDLV YISHRAFSGL NSLEQLTLEK
CNLTSIPTEA LSHLHGLIVL RLRHLNINAI RDYSFKRLYR
LKVLEISHWP YLDTMTPNCL YGLNLTSLSI THCNLTAVPY
LAVRHLVYLR FLNLSYNPIS TIEGSMLHEL LRLQEIQLVG
GQLAVVEPYA FRGLNYLRVL NVSGNQLTTL EESVFHSVGN
LETLILDSNP LACDCRLLWV FRRRWRLNFN RQQPTCATPE
FVQGKEFKDF PDVLLPNYFT CRRARIRDRK AQQVFVDEGH
TVQFVCRADG DPPPAILWLS PRKHLVSAKS NGRLTVFPDG
TLEVRYAQVQ DNGTYLCIAA NAGGNDSMPA HLHVRSYSPD
WPHQPNKTFA FISNQPGEGE ANSTRATVPF PFDIKTLIIA
TTMGFISFLG VVLFCLVLLF LWSRGKGNTK HNIEIEYVPR
KSDAGISSAD APRKFNMKM.

See, e.g., NCBI Reference Sequence: NP_001288115.1; UniProtkB/Swiss-Prot accession number Q96FE5.

### Anti-LINGO-1 Antibodies

The anti-LINGO-1 antibody or LINGO-1-binding fragment thereof used in the compositions and methods described herein binds to human LINGO-1.

In one embodiment, the antibody molecule is isolated, purified or recombinant. By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

As used herein, the term “antibody molecule” refers to a protein comprising at least one immunoglobulin variable domain sequence. The term antibody molecule includes, for example, full-length antibodies, mature antibodies, fragments, e.g., antigen-binding fragments of an antibody, derivatives, analogs, or variants of the antibodies disclosed herein, and any combination thereof.

The terms “fragment,” “variant,” “derivative” and “analog” when referring to LINGO-1 antibody molecules or antibody polypeptides include any polypeptides which retain at least some of the antigen-binding properties of the corresponding native antibody or polypeptide. Fragments of polypeptides include proteolytic fragments, as well as deletion fragments, in addition to specific antibody fragments discussed elsewhere herein. Variants of LINGO-1 antibody and antibody polypeptides include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Derivatives of LINGO-1 antibody molecules and antibody polypeptides are polypeptides which have been altered so as to exhibit additional features not found on the native polypeptide. Examples include fusion proteins.

In one embodiment, the anti-LINGO-1 antibody molecule is a fully human anti-LINGO-I monoclonal antibody engineered into an aglycosyl immunoglobulin G subclass 1 (IgG 1) framework to reduce effector function (also referred to herein as Li81). Histological and functional evaluations of LINGO-1 knock-out mice have been performed, and in vivo pharmacological activity of Li81 has been demonstrated in several animal models of demyelination. Li81 has been characterized in vitro and in vivo based on the evaluation of binding characteristics, biological activity, and pharmacological activity. The results of these studies indicate that Li81 has the following characteristics: (a) binds to LINGO-1 with similar high apparent affinity across human, monkey, rat and mouse; (b) is selective for LINGO-1 and does not bind the other LINGO family members, namely LINGO-2, LINGO-3, or LINGO-4; (c) enhances differentiation of primary rat, monkey, and human oligodendrocytes in vitro; (d) enhances axonal myelination in an in vitro rat dorsal root ganglion/OPC co-culture bioassay; (e) has reduced Fc (gamma) and complement effector functions compared to wild-type IgG1; and (f) is efficacious in animal models using biochemical and functional readouts (e.g., is efficacious in animal models when given in the presence of interferon beta, and is efficacious in animal model when given in the presence of corticosteroids). Remyelination activity has been demonstrated in the rat LPC model following systemic administration of Li81 at 100 mg/kg. Functional recovery in the rat MOG-EAE model has been demonstrated following weekly systemic administration of 3 mg/kg and 10 mg/kg Li81.

In one embodiment, an anti-LINGO-1 antibody includes a heavy chain comprising the amino acid sequence of SEQ ID NO:9, or a sequence substantially identical thereto (e.g., an amino acid sequence at least 80%, 85%, 90% or 95% identical thereto).

In other embodiments, an anti-LINGO-1 antibody includes a light chain comprising the amino acid sequence of SEQ ID NO:17, or a sequence substantially identical thereto (e.g., an amino acid sequence at least 80%, 85%, 90% or 95% identical thereto).

In some embodiments, the anti-LINGO-1 antibodies bind to a convex surface of the leucine-rich repeat (LRR) domain within LRRs 4-8 of LINGO-1. Furthermore, binding of the anti-LINGO-1 antibody to Lingo-1 interfered with the ability of LINGO-1 to oligomerize.

In some embodiments, the anti-LINGO-1 antibody or LINGO-1-binding fragment thereof used in the compositions and methods described herein comprises the three heavy chain variable domain complementarity-determining regions (CDRs) of an antibody referred to as Li81.

Thus, Li81 is an exemplary anti-LINGO-1 antibody that can be used in the compositions and methods described herein. Li81 is a fully human IgG1 monoclonal antibody that binds with sub-nanomolar affinity to human LINGO-1. The K_{D} value for the binding affinity of Li81 to the LINGO-1 ectodomain was found to be less than or equal to 20 pM, and the KD value of the Fab of Li81 for the LINGO-1 ectodomain was found to be less than or equal to 50 pM. Importantly, Li81 does not bind to other highly homologous members of the LINGO family, e.g., LINGO-2, LINGO-3, and LINGO-4.

In some embodiments, the anti-LINGO-1 antibody or LINGO-1-binding fragment thereof comprises the three light chain variable domain CDRs of Li81. In some embodiments, the anti-LINGO-1 antibody or LINGO-1-binding fragment thereof comprises the three heavy chain variable domain CDRs of Li81. In still other embodiments, the anti-LINGO-1 antibody or LINGO-1-binding fragment thereof comprises the three heavy chain variable domain CDRs and the three light chain variable domain CDRs of Li81. The CDRs can be based on any CDR definition in the art, e.g., the definitions of Kabat, Chothia, Chothia from Abysis, enhanced Chothia/AbM, or based on the contact definition. VH sequences (and CDR sequences therein) of Li81 and other anti-LINGO-1 antibodies are provided in Table 2 below.

TABLE 2
LINGO-1 Antibody VH Sequences

| Antibody | VH SEQUENCE | VH CDR1 | VH CDR2 | VH CDR3 |
|---|---|---|---|---|
| Li62 | EVQLLESGGGLVQPGGSLRLSCAASGFTFSIYPMFWVRQAP | IYPMF | WIGPSGGITK | EGHNDWYFDL |
| | GKGLEWVSWIGPSGGITKYADSVKGRFTISRDNSKNTLYLQ | (SEQ ID | YADSVKG | (SEQ ID |
| | MNSLRAEDTATYYCAREGHNDWYGFLWGRGTLVTVSS | NO: 2) | (SEQ ID | NO: 4) |
| | (SEQ ID NO: 1) | | NO: 3) | |
| Li62 | EVQLLESGGGLVQPGGSLRLSCAASGFTFSIYPMFWVRQAP | IYPMF | WIGPSGGITK | EGYYDWYFDQ |
| variant | GKGLEWVSWIGPSGGITKYADSVKGRFTISRDNSKNTLYLQ | (SEQ ID | YADSVKG | (SEQ ID |
| B06 | MNSLRAEDTATYYCAREGYYDWYFDQWGRGTLVTVSS | NO: 2) | (SEQ ID | NO: 50) |
| | (SEQ ID NO: 53) | | NO: 3) | |
| Li62 | EVQLLESGGGLVQPGGSLRLSCAASGFTFSIYPMFWVRQAP | IYPMF | WIGPSGGITK | EGQYDWYFDV |
| variant | GKGLEWVSWIGPSGGITKYADSVKGRFTISRDNSKNTLYLQ | (SEQ ID | YADSVKG | (SEQ ID |
| B12 | MNSLRAEDTATYYCAREGQYDWYGDVWGRGTLVTVSS | NO: 2) | (SEQ ID | NO: 18) |
| | (SEQ ID NO: 54) | | NO: 3) | |
| Li62 | EVQLLESGGGLVQPGGSLRLSCAASGFTFSIYPMFWVRQAP | IYPMF | WIGPSGGITK | EGDYDWYFDL |
| variant | GKGLEWVSWIGPSGGITYKADSVKGRFTISRDNSKNTLYLQ | (SEQ ID | YADSVKG | (SEQ ID |
| F06 | MNSLRAEDTATYYCAREGDYDWYFDLWGRGTLVTVSS | NO: 2) | (SEQ ID | NO: 19) |
| | (SEQ ID NO: 55) | | NO: 3) | |
| Li62 | EVQLLESGGGLVQPGGSLRLSCAASGFTFSIYPMFWVRQAP | IYPMF | WIGPSGGITK | EGQYDWYFEL |
| variant | GKGLEWVSWIGPSGGITKYADSVKGRFTISRDNSKNTLYLQ | (SEQ ID | YADSVKG | (SEQ ID |
| B01 | MNSLRAEDTATYYCAREGQYDWYFELWGRGTLVTVSS | NO: 2) | (SEQ ID | NO: 20) |
| | (SEQ ID NO: 56) | | NO: 3) | |
| Li62 | EVQLLESGGGLVQPGGSLRLSCAASGFTFSIYPMFWVRQAP | IYPMF | WIGPSGGITK | EADIDWFFDL |
| variant | GKGLEWVSWIGPSGGITKYADSVKGRFTISRDNSKNTLYLQ | (SEQ ID | YADSVKG | (SEQ ID |
| D09 | MNSLRAEDTATYYCAREADIDWFFDLWGRGTLVTVSS | NO: 2) | (SEQ ID | NO: 21) |
| | (SEQ ID NO: 57) | | NO: 3) | |
| Li62 | EVQLLESGGGLVQPGGSLRLSCAASGFTFSIYPMFWVRQAP | IYPMF | WIGPSGGITK | EGHYDWYFDL |
| variant | GKGLEWVSWIGPSGGITKYADSVKGRFTISRDNSKNTLYLQ | (SEQ ID | YADSVKG | (SEQ ID |
| D12 | MNSLRAEDTATYYCAREGHYDWYFDLWGRGTLVTVSS | NO: 2) | (SEQ ID | NO: 22) |
| | (SEQ ID NO: 58) | | NO: 3) | |
| Li62 | EVQLLESGGGLVQPGGSLRLSCAASGFTFSIYPMFWVRQAP | IYPMF | WIGPSGGITK | EGRYDWYFDP |
| variant | GKGLEWVSWIGPSGGITKYADSVKGRFTISRDNSKNTLYLQ | (SEQ ID | YADSVKG | (SEQ ID |
| F01 | MNSLRAEDTATYYCAREGRYDWYGDPWGRGTLVTVSS | NO: 2) | (SEQ ID | NO: 23) |
| | (SEQ ID NO: 59) | | NO: 3) | |
| Li62 | EVQLLESGGGLVQPGGSLRLSCAASGFTFSIYPMFWVRQAP | IYPMF | WIGPSGGITK | EGDYDWYFGL |
| variant | GKGLEWVSWIGPSGGITKYADSVKGRFTISRDNSKNTLYLQ | (SEQ ID | YADSVKG | (SEQ ID |
| F02 | MNSLRAEDTATYYCAREGDYDWYFGLWGRGTLVTVSS | NO: 2) | (SEQ ID | NO: 24) |
| | (SEQ ID NO: 60) | | NO: 3) | |
| Li62 | EVQLLESGGGLVQPGGSLRLSCAASGFTFSIYPMFWVRQAP | IYPMF | WIGPSGGITK | EGRYDWYFDL |
| variant | GKGLEWVSWIGPSGGITKYADSVKGRFTISRDNSKNTLYLQ | (SEQ ID | YADSVKG | (SEQ ID |
| F06 | MNSLRAEDTATYYCAREGRYDWYFDLWGRGTLVTVSS | NO: 2) | (SEQ ID | NO: 25) |
| | (SEQ ID NO: 61) | | NO: 3) | |
| Li62 | EVQLLESGGGLVQPGGSLRLSCAASGFTFSIYPMFWVRQAP | IYPMF | WIGPSGGITK | ESHIDRYFDL |
| variant | GKGLEWVSWIGPSGGITKYADSVKGRFTISRDNSKNTLYLQ | (SEQ ID | YADSVKG | (SEQ ID |
| F10 | MNSLRAEDTATYYCARESHIDRYFDLWGRGTLVTVSS | NO: 2) | (SEQ ID | NO: 26) |
| | (SEQ ID NO: 62) | | NO: 3) | |
| Li62 | EVQLLESGGGLVQPGGSLRLSCAASGFTFSIYPMFWVRQAP | IYPMF | WIGPSGGITK | EGQYDWYFDV |
| variant | GKGLEWVSWIGPSGGITKYADSVKGRFTISRDNSKNTLYLQ | (SEQ ID | YADSVKG | (SEQ ID |
| G08 | MNSLRAEDTATYYCAREGQYDWYFDVWGRGTLVTVSS | NO: 2) | (SEQ ID | NO: 27) |
| | (SEQ ID NO: 63) | | NO: 3) | |
| Li62 | EVQLLESGGGLVQPGGSLRLSCAASGFTFSIYPMFWVRQAP | IYPMF | WIGPSGGITK | EGHYNGYFDL |
| variant | GKGLEWVSWIGPSGGITKYADSVKGRFTISRDNSKNTLYLQ | (SEQ ID | YADSVKG | (SEQ ID |
| H08 | MNSLRAEDTATYYCAREGHYNGYFDLWGRGTLVTVSS | NO: 2) | (SEQ ID | NO: 28) |
| | (SEQ ID NO: 64) | | NO: 3) | |
| Li62 | EVQLLESGGGLVQPGGSLRLSCAASGFTFSIYPMFWVRQAP | IYPMF | WIGPSGGITK | EGYYDWYFDL |
| variant | GKGLEWVSWIGPSGGITKYADSVKGRFTISRDNSKNTLYLQ | (SEQ ID | YADSVKG | (SEQ ID |
| C10 | MNSLRAEDTATYYCAREGYYDWYFDLWGRGTLVTVSS | NO: 2) | (SEQ ID | NO: 29) |
| | (SEQ ID NO: 65) | | NO: 3) | |
| Li62 | EVQLLESGGGLVQPGGSLRLSCAASGFTFSIYPMFWVRQAP | IYPMF | WIGPSGGITK | EGTYDWYLDL |
| variant | GKGLEWVSWIGPSGGITKYADSVKGRFTISRDNSKNTLYLQ | (SEQ ID | YADSVKG | (SEQ ID |
| C02 | MNSLRAEDTATYYCAREGTYDWYLDLWGRGTLVTVSS | NO: 2) | (SEQ ID | NO: 30) |
| | (SEQ ID NO: 66) | | NO: 3) | |
| Li62 | EVQLLESGGGLVQPGGSLRLSCAASGFTFSIYPMFWVRQAP | IYPMF | WIGPSGGITK | EGYYDWYFEL |
| variant | GKGLEWVSWIGPSGGITKYADSVKGRFTISRDNSKNTLYLQ | (SEQ ID | YADSVKG | (SEQ ID |
| D05 | MNSLRAEDTATYYCAREGYYDWYFELWGRGTLVTVSS | NO: 2) | (SEQ ID | NO: 31) |
| | (SEQ ID NO: 67) | | NO: 3) | |
| Li62 | EVQLLESGGGLVQPGGSLRLSCAASGFTFSIYPMFWVRQAP | IYPMF | WIGPSGGITK | EGLIDWFFDQ |
| variant | GKGLEWVSWIGPSGGITKYADSVKGRFTISRDNSKNTLYLQ | (SEQ ID | YADSVKG | (SEQ ID |
| F02 | MNSLRAEDTATYYCAREGLIDWFFDLQWGRGTLVTVSS | NO: 2) | (SEQ ID | NO: 32) |
| | (SEQ ID NO: 68) | | NO: 3) | |
| Li62 | EVQLLESGGGLVQPGGSLRLSCAASGFTFSIYPMFWVRQAP | IYPMF | WIGPSGGITK | EGQFDWYFDL |
| variant | GKGLEWVSWIGPSGGITKYADSVKGRFTISRDNSKNTLYLQ | (SEQ ID | YADSVKG | (SEQ ID |
| C10 | MNSLRAEDTATYYCAREGQFDWYFDLWGRGTLVTVSS | NO: 2) | (SEQ ID | NO: 33) |
| | (SEQ ID NO: 69) | | NO: 3) | |
| Li62 | EVQLLESGGGLVQPGGSLRLSCAASGFTFSIYPMFWVRQAP | IYPMF | WIGPSGGITK | EGTYDWYFDL |
| variant | GKGLEWVSWIGPSGGITKYADSVKGRFTISRDNSKNTLYLQ | (SEQ ID | YADSVKG | (SEQ ID |
| H08 | MNSLRAEDTATYYCAREGTYDWYFDLWGRGTLVTVSS | NO: 2) | (SEQ ID | NO: 34) |
| | (SEQ ID NO: 70) | | NO: 3) | |
| Li81 | EVQLLESGGGLVQPGGSLRLSCAASGFTFSAYEMKWVRQAP | AYEMK | VIGPSGGFT | EGDNDAFDI |
| | GKGLEWVSVIGPSGGFTFYADSVKGRFTISRDNSKNTLYLQ | (SEQ ID | FYADSVKG | (SEQ ID |
| | MNSLRAEDTAVYYCATEGDNDAFDIWGQGTTVTVSS | NO: 6) | (SEQ ID | NO: 8) |
| | (SEQ ID NO: 5) | | NO: 7) | |
| Li81 | EVQLLESGGGLVQPGGSLRLSCAASGFTFSAYEMKWVRQAP | AYEMK | VIGPSGGFT | EGENDAFDV |
| variant | GKGLEWVSVIGPSGGFTFYADSVKGRFTISRDNSKNTLYLQ | (SEQ ID | FYADSVKG | (SEQ ID |
| F09 | MNSLRAEDTAVYYCATEGENDAFDVWGQGTTVTVSS | NO: 6) | (SEQ ID | NO: 35) |
| | (SEQ ID NO: 71) | | NO: 7) | |
| Li81 | EVQLLESGGGLVQPGGSLRLSCAASGFTFSAYEMKWVRQAP | AYEMK | VIGPSGGFT | EGDNDAYDT |
| variant | GKGLEWVSVIGPSGGFTFYADSVKGRFTISRDNSKNTLYLQ | (SEQ ID | FYADSVKG | (SEQ ID |
| G02 | MNSLRAEDTAVYYCATEGDNDAYDTWGQGTTVTVSS | NO: 6) | (SEQ ID | NO: 36) |
| | (SEQ ID NO: 72) | | NO: 7) | |
| Li81 | EVQLLESGGGLVQPGGSLRLSCAASGFTFSAYEMKWVRQAP | AYEMK | VIGPSGGFT | EGTNDAFDI |
| variant | GKGLEWVSVIGPSGGFTFYADSVKGRFTISRDNSKNTLYLQ | (SEQ ID | FYADSVKG | (SEQ ID |
| H03 | MNSLRAEDTAVYYCATEGTNDAFDIWGQGTTVTVSS | NO: 6) | (SEQ ID | NO: 37) |
| | (SEQ ID NO: 73) | | NO: 7) | |
| Li81 | EVQLLESGGGLVQPGGSLRLSCAASGFTFSAYEMKWVRQAP | AYEMK | VIGPSGGFT | EGDNDAFDS |
| variant | GKGLEWVSVIGPSGGFTFYADSVKGRFTISRDNSKNTLYLQ | (SEQ ID | FYADSVKG | (SEQ ID |
| A12 | MNSLRAEDTAVYYCATEGDNDAFDSWGQGTTVTVSS | NO: 6) | (SEQ ID | NO: 38) |
| | (SEQ ID NO: 74) | | NO: 7) | |
| Li81 | EVQLLESGGGLVQPGGSLRLSCAASGFTFSAYEMKWVRQAP | AYEMK | VIGPSGGFT | EGDNDAFDT |
| variant | GKGLEWVSVIGPSGGFTFYADSVKGRFTISRDNSKNTLYLQ | (SEQ ID | FYADSVKG | (SEQ ID |
| C02 | MNSLRAEDTAVYYCATEGDNDAFDTWGQGTTVTVSS | NO: 6) | (SEQ ID | NO: 39) |
| | (SEQ ID NO: 75) | | NO: 7) | |
| Li81 | EVQLLESGGGLVQPGGSLRLSCAASGFTFSAYEMKWVRQAP | AYEMK | VIGPSGGFT | EGDNDAYDR |
| variant | GKGLEWVSVIGPSGGFTFYADSVKGRFTISRDNSKNTLYLQ | (SEQ ID | FYADSVKG | (SEQ ID |
| C11 | MNSLRAEDTAVYYCATEGDNDAYDRWGQGTTVTVSS | NO: 6) | (SEQ ID | NO: 40) |
| | (SEQ ID NO: 76) | | NO: 7) | |
| Li81 | EVQLLESGGGLVQPGGSLRLSCAASGFTFSAYEMKWVRQAP | AYEMK | VIGPSGGFT | EGDNDVFDS |
| variant | GKGLEWVSVIGPSGGFTFYADSVKGRFTISRDNSKNTLYLQ | (SEQ ID | FYADSVKG | (SEQ ID |
| D11 | MNSLRAEDTAVYYCATEGDNDVFDSWGQGTTVTVSS | NO: 6) | (SEQ ID | NO: 41) |
| | (SEQ ID NO: 77) | | NO: 7) | |
| Li81 | EVQLLESGGGLVQPGGSLRLSCAASGFTFSAYEMKWVRQAP | AYEMK | VIGPSGGFT | EGDDDVFDM |
| variant | GKGLEWVSVIGPSGGFTFYADSVKGRFTISRDNSKNTLYLQ | (SEQ ID | FYADSVKG | (SEQ ID |
| E05 | MNSLRAEDTAVYYCATEGDDDVFDMWGQGTTVTVSS | NO: 6) | (SEQ ID | NO: 42) |
| | (SEQ ID NO: 78) | | NO: 7) | |
| Li81 | EVQLLESGGGLVQPGGSLRLSCAASGFTFSAYEMKWVRQAP | AYEMK | VIGPSGGFT | EGYNDAFDF |
| variant | GKGLEWVSVIGPSGGFTFYADSVKGRFTISRDNSKNTLYLQ | (SEQ ID | FYADSVKG | (SEQ ID |
| H04 | MNSLRAEDTAVYYCATEGYNDAFDFWGQGTTVTVSS | NO: 6) | (SEQ ID | NO: 43) |
| | (SEQ ID NO: 79) | | NO: 7) | |
| Li81 | EVQLLESGGGLVQPGGSLRLSCAASGFTFSAYEMKWVRQAP | AYEMK | VIGPSGGFT | EGDDDAYDM |
| variant | GKGLEWVSVIGPSGGFTFYADSVKGRFTISRDNSKNTLYLQ | (SEQ ID | FYADSVKG | (SEQ ID |
| B04 | MNSLRAEDTAVYYCATEGDDDAYDMWGQGTTVTVSS | NO: 6) | (SEQ ID | NO: 44) |
| | (SEQ ID NO: 80) | | NO: 7) | |
| Li81 | EVQLLESGGGLVQPGGSLRLSCAASGFTFSAYEMKWVRQAP | AYEMK | VIGPSGGFT | EQDYDTYDL |
| variant | GKGLEWVSVIGPSGGFTFYADSVKGRFTISRDNSKNTLYLQ | (SEQ ID | FYADSVKG | (SEQ ID |
| A02 | MNSLRAEDTAVYYCATEQDYDTYDLWGQGTTVTVSS | NO: 6) | (SEQ ID | NO: 45) |
| | (SEQ ID NO: 81) | | NO: 7) | |
| Li81 | EVQLLESGGGLVQPGGSLRLSCAASGFTFSAYEMKWVRQAP | AYEMK | VIGPSGGFT | EGDDDAFDT |
| variant | GKGLEWVSVIGPSGGFTFYADSVKGRFTISRDNSKNTLYLQ | (SEQ ID | FYADSVKG | (SEQ ID |
| B12 | MNSLRAEDTAVYYCATEGDDDAFDTWGQGTTVTVSS | NO: 6) | (SEQ ID | NO: 46) |
| | (SEQ ID NO: 82) | | NO: 7) | |
| Li81 | EVQLLESGGGLVQPGGSLRLSCAASGFTFSAYEMKWVRQAP | AYEMK | VIGPSGGFT | EADDDAFDI |
| variant | GKGLEWVSVIGPSGGFTFYADSVKGRFTISRDNSKNTLYLQ | (SEQ ID | FYADSVKG | (SEQ ID |
| H06 | MNSLRAEDTAVYYCATEADDDAFDIWGQGTTVTVSS | NO: 6) | (SEQ ID | NO: 47) |
| | (SEQ ID NO: 83) | | NO: 7) | |
| Li81 | EVQLLESGGGLVQPGGSLRLSCAASGFTFSAYEMKWVRQAP | AYEMK | VIGPSGGFT | EGENDAFDM |
| variant | GKGLEWVSVIGPSGGFTFYADSVKGRFTISRDNSKNTLYLQ | (SEQ ID | FYADSVKG | (SEQ ID |
| H08 | MNSLRAEDTAVYYCATEGENDAFDMWGQGTTVTVSS | NO: 6) | (SEQ ID | NO: 48) |
| | (SEQ ID NO: 84) | | NO: 7) | |
| Li81 | EVQLLESGGGLVQPGGSLRLSCAASGFTFSAYEMKWVRQAP | AYEMK | VIGPSGGFT | EGEYDTYDI |
| variant | GKGLEWVSVIGPSGGFTFYADSVKGRFTISRDNSKNTLYLQ | (SEQ ID | FYADSVKG | (SEQ ID |
| E07 | MNSLRAEDTAVYYCATEGEYDTYDIWGQGTTVTVSS | NO: 6) | (SEQ ID | NO: 49) |
| | (SEQ ID NO: 85) | | NO: 7) | |

VL sequences (and CDR sequences therein) of Li81 and other anti-LINGO-1 antibodies are provided in Table 3 below.

TABLE 3
LINGO-1 Antibody VL Sequences

| Antibody | VL SEQUENCE | VL CDR1 | VL CDR2 | VL CDR3 |
|---|---|---|---|---|
| Li62 | DIQMTQSPSFLSASVGDSVAITCRASQDISRYLAW | RASQDISRYLA | DASNLQT | QQYDTLHPS |
| | YQQRPGKAPKLLIYDASNLQTGVPSRFSGSGSGTD | (SEQ ID | (SEQ ID | (SEQ ID |
| | FTFTITSLQPEDFGTYYCQQYDTLHPSFGPGTTVD | NO: 10) | NO: 11) | NO: 12) |
| | IK (SEQ ID NO: 51) | | | |
| Li81 | DIQMTQSPATLSLSPGERATLSCRASQSVSSYLAW | RASQSVSSYLA | DASNRAT | QQRSNWPMYT |
| | YQQKPGQAPRLLIYDASNRATGIPARFSGSGSGTD | (SEQ ID | (SEQ ID | (SEQ ID |
| | FTLTISSLEPEDFAVYYCQQRSNWPMYTFGQGTKL | NO: 14) | NO: 15) | NO: 16) |
| | EIK (SEQ ID NO: 13) | | | |

In some embodiments, the anti-LINGO-1 antibody or LINGO-1-binding fragment thereof comprises a VH CDR1 comprising or consisting of the amino acid sequence set forth in SEQ ID NO:6, a VH CDR2 comprising or consisting of the amino acid sequence set forth in SEQ ID NO:7; and a VH CDR3 comprising or consisting of the amino acid sequence set forth in SEQ ID NO:8. In some embodiments, the anti-LINGO-1 antibody or LINGO-1-binding fragment thereof comprises a VL CDR1 comprising or consisting of the amino acid sequence set forth in SEQ ID NO:14, a VL CDR2 comprising or consisting of the amino acid sequence set forth in SEQ ID NO:15; and a VL CDR3 comprising or consisting of the amino acid sequence set forth in SEQ ID NO:16.

In some embodiments, the anti-LINGO-1 antibody or LINGO-1-binding fragment thereof comprises a VH CDR1 comprising or consisting of the amino acid sequence set forth in SEQ ID NO:6, a VH CDR2 comprising or consisting of the amino acid sequence set forth in SEQ ID NO:7; and a VH CDR3 comprising or consisting of the amino acid sequence set forth in SEQ ID NO:8; a VL CDR1 comprising or consisting of the amino acid sequence set forth in SEQ ID NO:14, a VL CDR2 comprising or consisting of the amino acid sequence set forth in SEQ ID NO:15; and a VL CDR3 comprising or consisting of the amino acid sequence set forth in SEQ ID NO:16.

In some embodiments, the anti-LINGO-1 antibody or LINGO-1-binding fragment thereof comprises or consists of the variable heavy chain (VH) of Li81. The VH of Li81 has the following amino acid sequence (VH-CDRs underlined):

(SEQ ID NO: 5)
EVQLLESGGGLVQPGGSLRLSCAASGFTFSAYEMKWVRQAPGKGLEWVSV
IGPSGGFTFYADSVKGRFTISRDNSKNTLYLQMNSLRAEDTAVYYCATEG
DNDAFDIWGQGTTVTVSS

In some embodiments, the anti-LINGO-1 antibody or LINGO-1-binding fragment thereof comprises or consists of the variable light chain (VL) of Li81. The VL of Li81 has the following amino acid sequence (VL-CDRs underlined):

(SEQ ID NO: 13)
DIQMTQSPATLSLSPGERATLSCRASQSVSSYLAWYQQKPGQAPRLLIYD
ASNRATGIPARFSGSGSGTDFTLTISSLEPEDFAVYYCQQRSNWPMYTFG
QGTKLEIK

An antibody consisting of the mature heavy chain (SEQ ID NO:9) and the mature light chain (SEQ ID NO:17) listed below is termed “Li81” as used herein.

Mature Li81 heavy chain (HC):
(SEQ ID NO: 9)
EVQLLESGGGLVQPGGSLRLSCAASGFTFSAYEMKWVRQAPGKGLEWVSV
IGPSGGFTFYADSVKGRFTISRDNSKNTLYLQMNSLRAEDTAVYYCATEG
DNDAFDIWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSA
YRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG.
Mature Li81 light chain (LC):
(SEQ ID NO: 17)
DIQMTQSPATLSLSPGERATLSCRASQSVSSYLAWYQQKPGQAPRLLIYD
ASNRATGIPARFSGSGSGTDFTLTISSLEPEDFAVYYCQQRSNWPMYTFG
QGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In the above-listed VH, VL, HC, and LC sequences, CDRs 1, 2, and 3 are based on the Kabat definition.

In certain embodiments of the methods and compositions disclosed herein, the anti-LINGO-1 antibody or LINGO-1-binding fragment thereof comprises a VH having the amino acid sequence set forth in SEQ ID NO:5. In certain embodiments of the methods and compositions disclosed herein, the anti-LINGO-1 antibody or LINGO-1-binding fragment thereof comprises a VL having the amino acid sequence set forth in SEQ ID NO:13. In certain embodiments of the methods and compositions disclosed herein, the anti-LINGO-1 antibody or LINGO-1-binding fragment thereof comprises a VH having the amino acid sequence set forth in SEQ ID NO:5 and a VL having the amino acid sequence set forth in SEQ ID NO:13. In certain embodiments of the methods and compositions disclosed herein, the anti-LINGO-1 antibody or LINGO-1-binding fragment thereof comprises a heavy chain having the amino acid sequence set forth in SEQ ID NO:9. In certain embodiments of the methods and compositions disclosed herein, the anti-LINGO-1 antibody or LINGO-1-binding fragment thereof comprises a light chain having the amino acid sequence set forth in SEQ ID NO:17. In other embodiments of the methods and compositions disclosed herein, the anti-LINGO-1 antibody or LINGO-1-binding fragment thereof comprises a heavy chain having the amino acid sequence set forth in SEQ ID NO:9 and a light chain having the amino acid sequence set forth in SEQ ID NO:17.

In some embodiments, the anti-LINGO-1 antibody or LINGO-1-binding fragment thereof selectively binds to LINGO-1 and comprises a HC that is at least 70%, 75%, 80%, 85%, 90%, 91%, 92%, 93%, 94%, 95%, 96%, 97%, 98%, 99% or more identical to the amino acid sequence of SEQ ID NO:9, or differs at least at 1 to 5 amino acid residues, but at fewer than 40, 30, 20, 15, or 10, residues, from SEQ ID NO:9. In one embodiment, the six CDRs are identical to the six CDRs of Li81 and any substitutions are made to the framework region.

In some embodiments, the anti-LINGO-1 antibody or LINGO-1-binding fragment thereof selectively binds to LINGO-1 and comprises a LC that is at least 70%, 75%, 80%, 85%, 90%, 91%, 92%, 93%, 94%, 95%, 96%, 97%, 98%, 99% or more identical to the amino acid sequence of SEQ ID NO:17, or differs at least at 1 to 5 amino acid residues, but at fewer than 40, 30, 20, 15, or 10, residues, from SEQ ID NO:17. In one embodiment, the six CDRs are identical to the six CDRs of Li81 and any substitutions are made to the framework region.

In certain embodiments, the anti-LINGO-1 antibody is an IgG antibody. In specific embodiments, the anti-LINGO-1 antibody has heavy chain constant region chosen from, e.g., IgG1, IgG2, IgG3, IgG4, IgM, IgA1, IgA2, IgD, and IgE. In one embodiment, the anti-LINGO-1 antibody is of the IgG1 isotype. In another embodiment, the anti-LINGO-1 antibody is of the IgG2 isotype. In yet another embodiment, the anti-LINGO-1 antibody is of the IgG3 isotype. In further embodiments, the anti-LINGO-1 antibody has a light chain constant region chosen from, e.g., a human kappa or human lambda light chain. In a certain embodiment, the anti-LINGO-1 antibody is an IgG1/human lambda antibody.

In some embodiments, the anti-LINGO-1 antibody is a full-length (whole) antibody or substantially full-length. The protein can include at least one, and preferably two, complete heavy chains, and at least one, and preferably two, complete light chains. In some embodiments, the anti-LINGO-1 antibody is a LINGO-1-binding fragment. In some instances, the LINGO-1-binding fragment is a Fab, a Fab′, an F(ab′)2, a Facb, an Fv, a single chain Fv (scFv), a sc(Fv)2, or a diabody.

The heavy chain and light chain of the antibodies disclosed herein may also include signal sequences. The signal sequences can be selected from those known in the art, for example, MDMRVPAQLLGLLLLWFPGSRC (SEQ ID NO:88) or MDMRVPAQLLGLLLLWLPGARC (SEQ ID NO:89).

Antibodies, such as Li81, or LINGO-1-binding fragments thereof can be made, for example, by preparing and expressing synthetic genes that encode the recited amino acid sequences or by mutating human germline genes to provide a gene that encodes the recited amino acid sequences. Moreover, this antibody and other anti-LINGO-1 antibodies can be produced, e.g., using one or more of the following methods.

### Anti-LINGO-1 Antibody Compositions

This disclosure also provides compositions (e.g., pharmaceutical compositions) comprising the anti-LINGO-1 antibody or LINGO-1-binding fragment thereof described herein. Note that compositions may also be referred to as formulations. For example, the anti-LINGO-1 compositions comprises an anti-LINGO-1 antibody or LINGO-1-binding fragment thereof comprising an immunoglobulin heavy chain variable domain (VH) and an immunoglobulin light chain variable domain (VL), wherein the VH comprises the H-CDRs and the VL comprises the L-CDRs of Li81. In certain instances, the heavy chain CDRs (H-CDRs) comprise or consist of the amino acid sequences set forth in SEQ ID NO: 6, SEQ ID NO:7, and SEQ ID NO:8; and the light chain CDRs (L-CDRs) comprise or consist of the amino acid sequences set forth in SEQ ID NO:14, SEQ ID NO:15, and SEQ ID NO:16. In some embodiments, the anti-LINGO-1 compositions comprises an anti-LINGO-1 antibody or LINGO-1-binding fragment thereof comprising (i) a VH comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:5; and (ii) a VL comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:13. In certain embodiments, the anti-LINGO-1 antibody compositions comprises an anti-LINGO-1 antibody comprising (i) a heavy chain comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:9; and (ii) a light chain comprising or consisting of an amino acid sequence that is at least 85%, 90%, 91%, 92%, 93%, 94%, 95%, 96%, 97%, 98%, 99%, or 100% identical to the amino acid sequence set forth in SEQ ID NO:17.

In certain embodiments, these compositions are high concentration anti-LINGO-1 antibody compositions. By “high concentration anti-LINGO-1 antibody composition” is meant a composition comprising anti-LINGO-1 antibodies or LINGO-1-binding fragments thereof at a concentration of greater than about 75 mg/ml and less than about 300 mg/ml. In certain instances, the high concentration anti-LINGO-1 antibody composition comprises anti-LINGO-1 antibodies or LINGO-1-binding fragments thereof at a concentration of about 100 mg/ml to about 275 mg/ml. In certain instances, the high concentration anti-LINGO-1 antibody composition comprises anti-LINGO-1 antibodies or LINGO-1-binding fragments thereof at a concentration of about 150 mg/ml to about 250 mg/ml. In certain instances, the high concentration anti-LINGO-1 antibody composition comprises anti-LINGO-1 antibodies or LINGO-1-binding fragments thereof at a concentration of about 175 mg/ml to about 225 mg/ml. In other instances, the high concentration anti-LINGO-1 antibody composition comprises anti-LINGO-1 antibodies or LINGO-1-binding fragments thereof at a concentration of about 180 mg/ml to about 220 mg/ml. In some instances, the high concentration anti-LINGO-1 antibody composition comprises anti-LINGO-1 antibodies or LINGO-1-binding fragments thereof at a concentration of about 190 mg/ml to about 210 mg/ml. In certain instances, the high concentration anti-LINGO-1 antibody composition comprises anti-LINGO-1 antibodies or LINGO-1-binding fragments thereof at a concentration of about 200 mg/ml.

A composition (e.g., a pharmaceutical composition) comprising an anti-LINGO-1 antibodies or LINGO-1-binding fragment described herein may be in any one of a variety of forms. These include, for example, liquid solutions (e.g., injectable and infusible solutions), dispersions, or suspensions. The preferred form can depend on the intended mode of administration and therapeutic application. In some embodiments, the route of administration of a composition comprising an anti-LINGO-1 antibodies or LINGO-1-binding fragment described herein is by any parenteral route including, without limitation, subcutaneous (SC/SQ), intraperitoneal (IP), intravenous (IV), intradermal (ID), and intramuscular (IM). In certain embodiments, a pharmaceutical composition described herein is in the form of a sterile injectable or infusible solution.

Sterile injectable solutions can be prepared by incorporating an antibody described herein in the required amount with one or a combination of ingredients, followed by filtered sterilization. Generally, dispersions are prepared by incorporating an antibody or antigen-binding fragment described herein into a sterile vehicle that contains a basic dispersion medium and the required other ingredients. In the case of sterile powders for the preparation of sterile injectable solutions, an exemplary method of preparation is vacuum drying and freeze drying that yields a powder of an antibody described herein plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants.

The anti-LINGO-1 antibody compositions (e.g., pharmaceutical compositions) (or compositions comprising a LINGO-1 binding fragment of an anti-LINGO-1 antibody) may additionally comprise one or more excipients. In some embodiments, the excipients may be selected from one or more of the following non-limited components: arginine hydrochloride (e.g., L-arginine hydrochloride), histidine (as free-base form of histidine, such as L-histidine, or histidine hydrochloride, such as L-histidine hydrochloride, or a combination of free-base form histidine and histidine hydrochloride), polysorbate 80, and proline (as free-base form of proline, such as L-proline, or proline hydrochloride, such as L-proline hydrochloride, or a combination of free-base form proline and proline hydrochloride) or methionine (as free-base form of methionine, such as L-methionine, or methionine hydrochloride, such as L-methionine hydrochloride, or a combination of free-base form methionine and methionine hydrochloride). In some embodiments, the anti-LINGO-1 antibody compositions (or compositions comprising a LINGO-1 binding fragment of an anti-LINGO-1 antibody) described herein do not include citrate (e.g., sodium citrate).

In one embodiment, the excipient in the anti-LINGO-1 antibody compositions (e.g., pharmaceutical compositions) (or compositions comprising a LINGO-1 binding fragment of an anti-LINGO-1 antibody) described herein buffers the pharmaceutical composition and/or increases the stability of the pharmaceutical composition and/or lowers/reduces the aggregation of the components (e.g., the antibody or antibody fragment) in the composition and/or lowers/reduces the viscosity of the antibody (or antibody fragment) in the composition as compared to the stability, aggregation and/or viscosity of the antibody in the pharmaceutical composition without that excipient.

In certain embodiments, the excipient in the anti-LINGO-1 antibody compositions (e.g., pharmaceutical compositions) (or compositions comprising a LINGO-1 binding fragment of an anti-LINGO-1 antibody) described herein is arginine, such as the free-base form of arginine (e.g., L-arginine), arginine hydrochloride (e.g., L-arginine hydrochloride) or a combination of the free-base form arginine (e.g., L-arginine) and arginine hydrochloride (e.g., L-arginine hydrochloride). In some embodiments, arginine is arginine hydrochloride (Arg HCl such as L-arginine hydrochloride). In some embodiments, the excipient in the anti-LINGO-1 antibody-containing compositions (or compositions comprising a LINGO-1 binding fragment of an anti-LINGO-1 antibody) described herein is arginine hydrochloride (Arg HCl, and not the free-base form of arginine, since arginine hydrochloride (Arg HCl) offers greater stability and viscosity benefits than the free-base form arginine. Arginine (e.g., the free-base form of arginine, such as L-arginine, or arginine hydrochloride, such as L-arginine hydrochloride, or a combination of free-base form arginine and arginine hydrochloride) can be included in the composition at a concentration of about 50 mM to about 300 mM, about 60 mM to about 250 mM, about 65 mM to about 200 mM, about 70 mM to about 175 mM, about 75 mM to about 170 mM, about 80 mM to about 160 mM, about 150 mM to about 165 mM, or about 160 mM. In certain embodiments arginine (e.g., arginine hydrochloride such as L-arginine hydrochloride) is present in the composition at a concentration of about 170 mM. In certain embodiments arginine (e.g., arginine hydrochloride such as L-arginine hydrochloride) is present in the composition at a concentration of about 165 mM. In a specific instance, arginine (e.g., arginine hydrochloride such as L-arginine hydrochloride) can be included in the composition at a concentration of about 160 mM. In certain embodiments arginine (e.g., arginine hydrochloride such as L-arginine hydrochloride) is present in the composition at a concentration of about 155 mM. In another specific instance, arginine (e.g., arginine hydrochloride such as L-arginine hydrochloride) can be included in the composition at a concentration of about 150 mM. In yet another specific instance, arginine (e.g., arginine hydrochloride such as L-arginine hydrochloride) can be included in the composition at a concentration of about 140 mM. In yet another specific instance, arginine (e.g., arginine hydrochloride such as L-arginine hydrochloride) can be included in the composition at a concentration of about 130 mM. In yet another specific instance, arginine (e.g., arginine hydrochloride such as L-arginine hydrochloride) can be included in the composition at a concentration of about 120 mM. In yet another specific instance, arginine (e.g., arginine hydrochloride such as L-arginine hydrochloride) can be included in the composition at a concentration of about 110 mM. In yet another specific instance, arginine (e.g., arginine hydrochloride such as L-arginine hydrochloride) can be included in the composition at a concentration of about 100 mM. In yet another specific instance, arginine (e.g., arginine hydrochloride such as L-arginine hydrochloride) can be included in the composition at a concentration of about 90 mM. In yet another specific instance, arginine (e.g., arginine hydrochloride such as L-arginine hydrochloride) can be included in the composition at a concentration of about 80 mM. In yet another specific instance, arginine (e.g., arginine hydrochloride such as L-arginine hydrochloride) can be included in the composition at a concentration of about 70 mM.

In certain embodiments, the excipient in the anti-LINGO-1 antibody compositions (e.g., pharmaceutical compositions) described herein is histidine, such as free-base form of histidine (e.g., L-histidine), histidine hydrochloride (e.g., L-histidine hydrochloride), or a combination of the free-base form of histidine and histidine hydrochloride. Histidine (e.g., a combination of the free-base form of histidine, such as L-histidine, or histidine hydrochloride, such as L-histidine hydrochloride) can be included in the composition at a concentration of about 0.1 mM to about 100 mM, about 1.0 mM to about 80 mM, about 5.0 mM to about 60 mM, about 10 mM to about 45 mM, about 15 mM to about 30 mM, about 15 mM to about 25 mM, about 18 mM to about 22 mM, or about 20 mM. In certain embodiments, histidine (e.g., a combination of the free-base form of histidine and/or histidine hydrochloride) is present in the composition at a concentration of about 20 mM. In certain embodiments, histidine (e.g., free-base form such as L-histidine), histidine hydrochloride (e.g., L-histidine hydrochloride), or a combination of the free-base form of histidine and histidine hydrochloride is included in the composition in a ratio that achieves the target pH of the composition. In some embodiments, the target pH of the composition is between about pH 6.0 and pH 7.0 (e.g., pH 6.5). Thus, when a composition is said to contain 20 mM histidine, it is understood that the composition contains 20 mM of histidine (e.g., free-base form histidine such as L-histidine), histidine hydrochloride, or a combination of free-base form histidine and histidine hydrochloride (e.g., L-histidine hydrochloride), with the amounts of free-base form of histidine and histidine hydrochloride varying to achieve the desired pH of the combination (e.g., pH 6.5).

In certain embodiments, the excipient in the anti-LINGO-1 antibody compositions (e.g., pharmaceutical compositions) described herein is methionine, such as the free-base form of methionine (e.g., L-methionine), methionine hydrochloride (e.g., methionine hydrochloride), or a combination of the free-base form methionine and methionine-HCl. In some embodiments, methionine is the free-base form of methionine (e.g., L-methionine). Methionine (e.g., the free-base form of methionine) can be included in the composition at a concentration of about 0.1 mM to about 50 mM, about 1.0 mM to about 37.5 mM, about 5.0 mM to about 25 mM, about 7.5 mM to about 20 mM, about 8 mM to about 15 mM, about 9 mM to about 12 mM, or about 10 mM. In certain embodiments, methionine (e.g., the free-base form of methionine such as L-methionine) is present in the composition at a concentration of about 10 mM.

In certain embodiments, the excipient in the anti-LINGO-1 antibody compositions (e.g., pharmaceutical compositions) described herein is proline, such as the free-base form of proline (e.g., L-proline), proline hydrochloride (e.g., L-proline hydrochloride), or a combination of the free-base form proline and proline-HCl. In some embodiments, proline is the free-base form of proline. Proline (e.g., the free-base form of proline, such as L-proline) can be included in the composition at a concentration of about 50 mM to about 300 mM, about 100 mM to about 200 mM, about 125 mM to about 175 mM, about 150 mM to about 165 mM, or about 160 mM. In certain embodiments, proline (e.g., the free-base form of proline, such as L-proline) is present in the composition at a concentration of about 160 mM.

In some embodiments, the anti-LINGO-1 antibody compositions (or compositions comprising a LINGO-1 binding fragment of an anti-LINGO-1 antibody) described herein do not include citrate. In some embodiments, the anti-LINGO-1 antibody compositions (or compositions comprising a LINGO-1 binding fragment of an anti-LINGO-1 antibody) described herein do not include glutamate. In some embodiments, the anti-LINGO-1 antibody compositions (or compositions comprising a LINGO-1 binding fragment of an anti-LINGO-1 antibody) described herein do not include trehalose. In some embodiments, the anti-LINGO-1 antibody compositions (or compositions comprising a LINGO-1 binding fragment of an anti-LINGO-1 antibody) described herein do not include sucrose. In some embodiments, the anti-LINGO-1 antibody compositions (or compositions comprising a LINGO-1 binding fragment of an anti-LINGO-1 antibody) described herein do not include calcium chloride. In some embodiments, the anti-LINGO-1 antibody compositions (or compositions comprising a LINGO-1 binding fragment of an anti-LINGO-1 antibody) described herein do not include lysine.

Antibody product manufacturing is a complex process that can involve several steps such as, e.g., drug substance and bulk formulation, filtration, shipping, pooling, filling, lyophilization, inspections, packaging, and storage. During these steps, antibodies may be subjected to many different forms of stresses, e.g., agitation, temperature, light exposure, and oxidation. These types of stresses can lead to denaturation and aggregation of the antibody, which compromise the product quality and can even lead to loss of a production batch. Agitation is one of the common physical stresses that antibody therapeutics are subjected to during the course of the manufacturing process. Agitation occurs, e.g., during mixing, ultrafiltration/diafiltration, pumping, shipping, and filling. To protect the antibody composition against agitation-induced stress, the composition may include a polysorbate. In certain embodiments, the composition comprises polysorbate-80 at a concentration of about 0.01% (w/v) to about 0.5% (w/v), about 0.01% (w/v) to about 0.1% (w/v), about 0.01% (w/v) to about 0.09% (w/v), about 0.02% (w/v) to about 0.08% (w/v), about 0.03% (w/v) to about 0.07% (w/v), about 0.04% (w/v) to about 0.07% (w/v), about 0.04% (w/v) to about 0.06% (w/v), or about 0.05% (w/v). In some embodiments, the composition comprises polysorbate-80 at a concentration of about 0.01% (w/v), about 0.02% (w/v), about 0.03% (w/v), about 0.04% (w/v), about 0.05% (w/v), about 0.06% (w/v), about 0.07% (w/v), about 0.08%, about 0.09%, or about 0.1% (w/v).

In some embodiments, the composition comprises more than 0.03% (weight per volume) polysorbate 80. For example, in some embodiments, the composition comprises 0.04% (w/v) polysorbate 80. In a particular embodiment, the composition comprises polysorbate-80 at a concentration of 0.05% (w/v) at pH 6.5.

In certain embodiments, the antibody composition comprises acetate as the buffering agent. In certain embodiments, the composition comprises acetate at a concentration of about 5 mM to about 50 mM, about 5 mM to about 40 mM, about 5 mM to about 35 mM, about 5 mM to about 30 mM, about 5 mM to about 25 mM, about 10 mM to about 50 mM, about 10 mM to about 40 mM, about 10 mM to about 30 mM, about 10 mM to about 25 mM, about 15 mM to about 50 mM, about 15 mM to about 40 mM, about 15 mM to about 30 mM, or about 15 mM to about 25 mM. In certain embodiments, the composition comprises acetate at a concentration of about 5 mM to about 35 mM. In certain embodiments, the composition comprises acetate at a concentration of about 10 mM to about 30 mM. In some embodiments, the composition comprises acetate at a concentration of about 5 mM, about 10 mM, about 15 mM, about 20 mM, about 25 mM, about 30 mM, or about 35 mM. In a particular embodiment, the composition comprises acetate at a concentration of about 20 mM.

In certain embodiments, the antibody composition comprises succinate as the buffering agent. In certain embodiments, the composition comprises succinate at a concentration of about 5 mM to about 50 mM, about 5 mM to about 40 mM, about 5 mM to about 35 mM, about 5 mM to about 30 mM, about 5 mM to about 25 mM, about 10 mM to about 50 mM, about 10 mM to about 40 mM, about 10 mM to about 30 mM, about 10 mM to about 25 mM, about 15 mM to about 50 mM, about 15 mM to about 40 mM, about 15 mM to about 30 mM, or about 15 mM to about 25 mM. In certain embodiments, the composition comprises succinate at a concentration of about 5 mM to about 35 mM. In certain embodiments, the composition comprises succinate at a concentration of about 10 mM to about 30 mM. In some embodiments, the composition comprises succinate at a concentration of about 5 mM, about 10 mM, about 15 mM, about 20 mM, about 25 mM, about 30 mM, or about 35 mM. In a particular embodiment, the composition comprises succinate at a concentration of about 20 mM.

The pH of the anti-LINGO-1 antibody composition can be from about 5.8 to about 7.2. In certain cases, the pH of the antibody composition can be about 6.0 to about 7.0. In certain instances, the pH of the antibody composition is about 6.0, about 6.1, about 6.2, about 6.3, about 6.4, about 6.5, about 6.5, about 6.7, about 6.8, about 6.9, or about 7.0. In a particular embodiment, the pH of the antibody composition is about 6.5.

In certain instances, the anti-LINGO-1 antibody compositions comprise arginine (e.g., the free-base form of arginine, such as L-arginine, or arginine hydrochloride, such as L-arginine hydrochloride, or a combination of free-base form arginine and arginine hydrochloride). In other instances, the anti-LINGO-1 antibody compositions comprise arginine (e.g., the free-base form of arginine, such as L-arginine, or arginine hydrochloride, such as L-arginine hydrochloride, or a combination of free-base form arginine and arginine hydrochloride) and histidine (as free-base form of histidine, such as L-histidine, or histidine hydrochloride, such as L-histidine hydrochloride, or a combination of free-base form histidine and histidine hydrochloride). In other instances, the anti-LINGO-1 antibody compositions comprise arginine (e.g., the free-base form of arginine, such as L-arginine, or arginine hydrochloride, such as L-arginine hydrochloride, or a combination of free-base form arginine and arginine hydrochloride), histidine (as free-base form of histidine, such as L-histidine, or histidine hydrochloride, such as L-histidine hydrochloride, or a combination of free-base form histidine and histidine hydrochloride), and methionine (e.g., the free-base form of methionine, such as L-methionine, or methionine hydrochloride, such as L-methionine hydrochloride, or a combination of free-base form methionine and methionine hydrochloride), and polysorbate 80. In yet other instances, the anti-LINGO-1 antibody compositions comprise arginine (as, e.g., free-base form of arginine, such as L-arginine, or arginine hydrochloride, such as L-arginine hydrochloride, or a combination of free-base form arginine and arginine hydrochloride), histidine (as, e.g., free-base form of histidine, such as L-histidine, or histidine hydrochloride, such as L-histidine hydrochloride, or a combination of free-base form histidine and histidine hydrochloride), proline (as, e.g., free-base form of proline, such as L-proline, or proline hydrochloride, such as L-proline hydrochloride, or a combination of free-base form proline and proline hydrochloride), and polysorbate 80.

In certain embodiments, the anti-LINGO-1 antibody compositions comprise arginine hydrochloride (as, e.g., L-arginine hydrochloride at, e.g., about 160 mM), histidine (as free-base form of histidine, such as L-histidine, or histidine hydrochloride, such as L-histidine hydrochloride, or a combination of free-base form histidine and histidine hydrochloride at, e.g., about 20 mM), methionine (as free-base form of methionine, such as L-methionine, or methionine hydrochloride, such as L-methionine hydrochloride, or a combination of free-base form methionine and methionine hydrochloride at, e.g., about 10 mM) and polysorbate 80 (e.g., about 0.05% w/v), and has a pH of about 6.0 to about 6.8 (e.g., pH of about 6.5). In some embodiments, the anti-LINGO-1 antibody compositions comprise arginine hydrochloride (as, e.g., L-arginine hydrochloride at, e.g., about 80 mM), histidine (as free-base form of histidine, such as L-histidine, or histidine hydrochloride, such as L-histidine hydrochloride, or a combination of free-base form histidine and histidine hydrochloride at, e.g., about 20 mM), proline (as free-base form of proline, such as L-proline, or proline hydrochloride, such as L-proline hydrochloride, or a combination of free-base form proline and proline hydrochloride at, e.g., 160 mM), and polysorbate (e.g., about 0.05% w/v), and has a pH of about 6.0 to about 6.8 (e.g., a pH of about 6.5). In all of these embodiments, the anti-LINGO-1 antibody is present at a concentration of about 50 mg/ml to about 300 mg/ml. In one instance, the anti-LINGO-1 antibody is present at a concentration of about 175 mg/ml. In one instance, the anti-LINGO-1 antibody is present at a concentration of about 200 mg/ml. In another instance, the anti-LINGO-1 antibody is present at a concentration of about 220 mg/ml.

### Methods of Producing Antibodies

Standard molecular biology techniques are used to prepare the recombinant expression vector(s), transfect the host cells, select for transformants, culture the host cells, and recover the antibody.

Anti-LINGO-1 antibodies or LINGO-1-binding fragments may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide encoding the antibody is constructed, introduced into an expression vector, and then expressed in suitable host cells. Polynucleotides encoding an anti-LINGO-1 antibody comprising the VH and/or VL, HC and/or LC of the anti-LINGO-1 antibodies described herein would be readily envisioned by the ordinarily skilled artisan. Standard molecular biology techniques are used to prepare the recombinant expression vector, transfect the host cells, select for transformants, culture the host cells and recover the antibody.

If the anti-LINGO-1 antibodies or LINGO-1-binding fragments is to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell may be a mammary epithelial cell. In a specific embodiment, the mammalian cell is a CHO-DG44I cell. In another example, the mammalian cell is a CHO-Kl cell.

In an exemplary system for antibody expression, a recombinant expression vector encoding both the antibody heavy chain and the antibody light chain of an anti-LINGO-1 antibody (e.g., Li81) is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Methods of Treatment

The anti-LINGO-1 antibody compositions described herein (e.g., Li81) can be used for the prophylactic and therapeutic treatment of a CNS demyelinating disease in a subject (e.g., human subject) in need thereof.

The anti-LINGO-1 antibody (e.g., Li81) or LINGO-1-binding fragment thereof described above can be administered to a subject, e.g., a human subject, at different doses. The anti-LINGO-1 antibody (e.g., Li81) or LINGO-1-binding fragment thereof can be administered as a fixed dose (i.e., independent of the weight of the patient), or in a mg/kg dose (i.e., a dose which varies based on the weight of the subject). Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. The treatment can continue for days, weeks, months or even years.

In one embodiment, for treating an indication described herein, the dosage of the anti-LINGO-1 antibody (e.g., Li81) or LINGO-1-binding fragment thereof is a fixed dose of 750 mg.

The fixed dose described above may each be administered daily, every week, every 2 weeks, every 4 weeks, every 6 weeks, every 8 weeks, monthly, biweekly, weekly, or daily, as appropriate, over a period of time to encompass at least 2 doses, 3 doses, 4 doses, 5 doses, 6 doses, 7 doses, 8 doses, 9 doses, 10 doses, 12 doses, 14 doses, 16 doses, 18 doses, 20 doses, 22 doses, 24 doses or more. In some embodiments, a fixed dose of 750 mg is administered parenterally once every 4 weeks.

In certain embodiments, a fixed dose of 750 mg of the anti-LINGO-1 antibody or LINGO-1-binding fragment thereof is administered to a human subject every 4 weeks for a period of time determined to be beneficial for the subject by her/his healthcare provider. In some instances a fixed dose of 750 mg of the anti-LINGO-1 antibody or LINGO-1-binding fragment thereof is administered to a human subject every 4 weeks. In some embodiments, the subject is administered at least 4, at least 5, at least 6, at least 7, at least 8, at least 9, or at least 10 doses of the fixed dose of 750 mg of the anti-LINGO-1 antibody or LINGO-1-binding fragment thereof. In some embodiments, the subject is administered 4, 5, 6, 7, 8, 9, or 10 doses of the fixed dose of 750 mg of the anti-LINGO-1 antibody or LINGO-1-binding fragment thereof. In some instances, the subject is administered 2 to 24, 2 to 20, 2 to 18, 2 to 16, 2 to 14, 2 to 12, 2 to 10, or 2 to 8 doses of the fixed dose of 50 mg of the anti-LINGO-1 antibody or LINGO-1-binding fragment thereof.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual. A therapeutically effective amount is also one in which any toxic, or detrimental effects, of the composition is outweighed by the therapeutically beneficial effects. In one embodiment, the therapeutically effective amount of the anti-LINGO-1 antibody or LINGO-1-binding fragment thereof is 750 mg administered once every 4 weeks.

The route and/or mode of administration of the anti-LINGO-1 antibody or LINGO-1-binding fragment thereof can be tailored for the individual subject. For many applications, the route of administration is one of: subcutaneous injection (SC), intravenous injection or infusion (IV), intraperitoneal administration (IP), or intramuscular injection. In one embodiment, the route of administration is subcutaneous. In another embodiment, the route of administration is intravenous.

It should be noted that the high concentration anti-LINGO-1 antibody compositions described herein can be administered directly, or can be diluted (e.g., in physiological saline) prior to administration. For example, if a dosage of 750 mg anti-LINGO-1 antibody is to be administered intravenously, and the high concentration anti-LINGO-1 antibody composition contains 200 mg/ml of an anti-LINGO-1 antibody (e.g., the Li81 antibody described herein), then 3.75 ml of the 200 mg/ml solution can simply be added to a 100 ml bag of physiological saline solution (e.g., 0.9% w/v NaCl) and that volume (i.e., 103.75 ml volume) can be administered intravenously.

It should also be noted that where a dosage of 750 mg anti-LINGO-1 antibody is to be administered with an undiluted high concentration anti-LINGO-1 antibody composition (e.g., subcutaneously), the full dosage can be split into multiple injections to achieve the full dosage. For example, for subcutaneous injections, particularly self-administered by the patient, it may be desirable for the injection volume to be less than 1.5 ml to minimize injection site pain. If the full dosage of anti-LINGO-1 antibody is 750 mg, and the high concentration anti-LINGO-1 antibody composition contains 200 mg/ml of an anti-LINGO-1 antibody, the full dosage can be administered at three injection sites, with 1.25 ml at each site (i.e., 250 mg anti-LINGO-1 antibody administered per injection site). Of course, in some embodiments, where the patient can tolerate an volume of 1.875 ml at a single injection site, the full dosage of 750 mg can be achieved with two 1.875 ml injections of a 200 mg/ml anti-LINGO-1 antibody composition at two different injection site.

Pharmaceutical compositions that comprise the anti-LINGO-1 antibody or LINGO-1-binding fragment thereof alone or in combination with non-LINGO-1 antibody agent(s) (e.g., an immunomodulatory agent such as natalizumab or interferon 1 beta) can be administered with a medical device. The device can be designed with features such as portability, room temperature storage, and ease of use so that it can be used in emergency situations, e.g., by an untrained subject or by emergency personnel in the field, removed to medical facilities and other medical equipment. The device can include, e.g., one or more housings for storing pharmaceutical preparations that include the anti-LINGO-1 antibody or LINGO-1-binding fragment thereof, and can be configured to deliver one or more unit doses of the blocking agent.

For example, the pharmaceutical composition can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

In one embodiment, the anti-LINGO-1 antibody or LINGO-1-binding fragment thereof is administered to a human subject with a syringe. In another embodiment, the anti-LINGO-1 antibody or LINGO-1-binding fragment thereof is administered to a human subject with a pump for subcutaneous delivery. In some embodiments, the anti-LINGO-1 antibody or LINGO-1-binding fragment thereof is administered to a human subject with an autoinjector. In other embodiments, the anti-LINGO-1 antibody or LINGO-1-binding fragment thereof is administered to a human subject with a subcutaneous large volume injector.

The disclosure also provides a syringe comprising a sterile preparation of the pharmaceutical compositions comprising an anti-LINGO-1 antibody (e.g., Li81) or LINGO-1-binding fragment thereof, as described above. As used herein, the term “syringe” collectively includes pumps, syringes, and injectors (e.g., autoinjector, subcutaneous large volume injector) that are able to administer a volume of a composition to a subject. The syringe can be adapted for subcutaneous administration of the anti-LINGO-1 antibody or LINGO-1-binding fragment thereof. In some cases, the syringe or pump delivers a fixed doses(s) (e.g., 750 mg) of the anti-LINGO-1 antibody or LINGO-1-binding fragment thereof.

Thus, some embodiments, the invention provides a kit comprising at least two syringes (e.g., adapted for subcutaneous administration) comprising a sterile preparation of the pharmaceutical composition comprising an anti-LINGO-1 antibody (e.g., Li81) or LINGO-1-binding fragment thereof, as described above, where the full dosage of the anti-LINGO-1 antibody (or LINGO-1 binding fragment thereof) comprised within the at least two syringes is a fixed dosage, such as 750 mg of the anti-LINGO-1 antibody. In some embodiments, where the composition comprises 200 mg/ml of an anti-LINGO-1 antibody, the kit provides three syringes, each of which contains a volume of 1.25 ml of the composition. In some embodiments, where the composition comprises 200 mg/ml of an anti-LINGO-1 antibody, the kit provides two syringes, each of which contains a volume of 1.875 ml of the composition.

In some embodiments, the kit further comprises an immunomodulatory agent, such as one of the immunomodulatory agents described below, for example, a syringe or an orally ingestible formulation containing an appropriate dosage of an immunomodulatory agent. The kit may further comprise, for example, instructions for administering the composition comprising an anti-LINGO-1 antibody and, optionally, instructions for administering the immunomodulatory agent.

### Immunomodulatory Agents

Several immunomodulatory agents are presently used to modify the course of multiple sclerosis in patients, and may be administered together with a composition comprising an anti-LINGO-1 antibody or LINGO-1-binding fragment thereof. Such agents include, but are not limited to, an IFN-β 1 molecule; a polymer of glutamic acid, lysine, alanine and tyrosine, e.g., glatiramer; an antibody or fragment thereof against alpha-4 integrin, e.g., natalizumab; an anthracenedione molecule, e.g., mitoxantrone; a fingolimod, e.g., FTY720; a fumarate, e.g., dimethyl fumarate, diroximel fumarate, or monomethyl fumarate, e.g., an oral fumarate, e.g., dimethyl fumarate, diroximel fumarate, or monomethyl fumarate; an antibody to the alpha subunit of the IL-2 receptor of T cells (CD25), e.g., daclizumab; an antibody against CD52, e.g., alemtuzumab; an inhibitor of a dihydroorotate dehydrogenase, e.g., teriflunomide; an antibody to CD20, e.g., ocrelizumab; and a corticosteroid. The reparative agents disclosed herein can be used in combination with any of these agents.

Exemplary immunomodulatory agents are described in more detail as follows.

### IFNβ Agents (Beta Interferons)

One known therapy for MS includes treatment with interferon beta. Interferons (IFNs) are natural proteins produced by the cells of the immune systems of most animals in response to challenges by foreign agents such as viruses, bacteria, parasites and tumor cells. Interferons belong to the large class of glycoproteins known as cytokines. Interferon beta has 165 amino acids. Interferons alpha and beta are produced by many cell types, including T-cells and B-cells, macrophages, fibroblasts, endothelial cells, osteoblasts and others, and stimulate both macrophages and NK cells. Interferon gamma is involved in the regulation of immune and inflammatory responses. It is produced by activated T-cells and Th1 cells.

Several different types of interferon are now approved for use in humans. Interferon alpha (including forms interferon alpha-2a, interferon alpha-2b, and interferon alfacon-1) was approved by the United States Food and Drug Administration (FDA) as a treatment for Hepatitis C. There are two currently FDA-approved types of interferon beta. Interferon beta 1a (Avonex®) is identical to interferon beta found naturally in humans, and interferon beta 1b (Betaseron®) differs in certain ways from interferon beta 1a found naturally in humans, including that it contains a serine residue in place of a cysteine residue at position 17. Other uses of interferon beta have included treatment of AIDS, cutaneous T-cell lymphoma, Acute Hepatitis C (non-A, non-B), Kaposi's sarcoma, malignant melanoma, hairy cell leukemia, and metastatic renal cell carcinoma.

IFNβ agents can be administered to the subject by any method known in the art, including systemically (e.g., orally, parenterally, subcutaneously, intravenously, rectally, intramuscularly, intravitreally, intraperitoneally, intranasally, transdermally, or by inhalation or intracavitary installation). Typically, the IFNβ agents are administered subcutaneously, or intramuscularly.

IFNβ agents can be used to treat those subjects determined to be “responders” using the methods described herein. In one embodiment, the IFNβ agents are used as a monotherapy (i.e., as a single “disease modifying therapy”) although the treatment regimen can further comprise the use of “symptom management therapies” such as antidepressants, analgesics, anti-tremor agents, etc. In one embodiment, the IFNβ agent is an IFNβ-1A agent (e.g., Avonex®, Rebif®). In another embodiment, the INFO agent is an INFO-1B agent (e.g., Betaseron®, Betaferon®, Extavia®).

Avonex®, an Interferon β-1a, is indicated for the treatment of patients with relapsing forms of MS that are determined to be responders using the methods described herein to slow the accumulation of physical disability and decrease the frequency of clinical exacerbations. Avonex® (Interferon beta-1a) is a 166 amino acid glycoprotein with a predicted molecular weight of approximately 22,500 daltons. It is produced by recombinant DNA technology using genetically engineered Chinese Hamster Ovary cells into which the human interferon beta gene has been introduced. The amino acid sequence of Avonex® is identical to that of natural human interferon beta. The recommended dosage of Avonex® (Interferon beta-1a) is 30 mcg injected intramuscularly once a week. Avonex® is commercially available as a 30 mcg lyophilized powder vial or as a 30 mcg prefilled syringe.

Interferon beta 1a (Avonex®) is identical to interferon beta found naturally in humans (AVONEX®, i.e., Interferon beta Ia (SwissProt Accession No. P01574 and gi:50593016). The sequence of interferon beta is:

(SEQ ID NO: 90)
MTNKCLLQIALLLCFSTTALSMSYNLLGFLQRSSNFQCQKLLWQLNGRLE
YCLKDRMNFDIPEEIKQLQQFQKEDAALTIYEMLQNIFAIFRQDSSSTGW
NETIVENLLANVYHQINHLKTVLEEKLEKEDFTRGKLMSSLHLKRYYGRI
LHYLKAKEYSHCAWTIVRVEILRNFYFINRLTGYLRN.

Methods for making Avonex® are known in the art.

Treatment of responders identified using the methods described herein further contemplates that compositions (e.g., IFN beta 1 a molecules) having biological activity that is substantially similar to that of AVONEX® will permit successful treatment similar to treatment with AVONEX® when administered in a similar manner. Such other compositions include, e.g., other interferons and fragments, analogues, homologues, derivatives, and natural variants thereof with substantially similar biological activity. In one embodiment, the INFO agent is modified to increase one or more pharmacokinetic properties. For example, the INFO agent can be a modified form of interferon 1a to include a pegylated moiety. PEGylated forms of interferon beta 1a are described in, e.g., Baker, D. P. et al. (2006) Bioconjug Chem 17(1):179-88; Arduini, R M et al. (2004) Protein Expr Purif 34(2):229-42; Pepinsky, R B et al. (2001) J. Pharmacol. Exp. Ther. 297(3):1059-66; Baker, D. P. et al. (2010) J Interferon Cytokine Res 30(10):777-85 (all of which are incorporated herein by reference in their entirety, and describe a human interferon beta 1a modified at its N-terminal alpha amino acid to include a PEG moiety, e.g., a 20 kDa mPEG-O-2-methylpropionaldehyde moiety). Pegylated forms of IFN beta 1a can be administered by, e.g., injectable routes of administration (e.g., subcutaneously).

Rebif® is also an Interferon β-1a agent, while Betaseron®, Betaferon®, and Extavia® are Interferon β-1b agents. Both Rebif® and Betaseron® are formulated for administration by subcutaneous injection.

Dosages of IFNβ agents to administer can be determined by one of skill in the art, and include clinically acceptable amounts to administer based on the specific interferon-beta agent used. For example, AVONEX® is typically administered at 30 microgram once a week via intramuscular injection. Other forms of interferon beta 1a, specifically REBIF®, is administered, for example, at 22 microgram three times a week or 44 micrograms once a week, via subcutaneous injection. Interferon beta-1A can be administered, e.g., intramuscularly, in an amount of between 10 and 50 μg. For example, AVONEX® can be administered every five to ten days, e.g., once a week, while Rebif® can be administered three times a week.

### Anti-VLA4 Antibody (e.g., Natalizumab (Tysabri®))

Anti-VLA4 antibodies (e.g., Natalizumab) inhibit the migration of leukocytes from the blood to the central nervous system. These antibodies bind to VLA-4 (also called a4β1) on the surface of activated T-cells and other mononuclear leukocytes. They can disrupt adhesion between the T-cell and endothelial cells, and thus prevent migration of mononuclear leukocytes across the endothelium and into the parenchyma. As a result, the levels of pro-inflammatory cytokines can also be reduced. Natalizumab can decrease the number of brain lesions and clinical relapses and accumulation of disability in patients with relapse remitting multiple sclerosis and relapsing secondary-progressive multiple sclerosis.

Natalizumab and related VLA-4 binding antibodies are described, e.g., in U.S. Pat. No. 5,840,299. Monoclonal antibodies 21.6 and HP1/2 are exemplary murine monoclonal antibodies that bind VLA-4. Natalizumab is a humanized version of murine monoclonal antibody 21.6 (see, e.g., U.S. Pat. No. 5,840,299). A humanized version of HP 1/2 has also been described (see, e.g., U.S. Pat. No. 6,602,503). Several additional VLA-4 binding monoclonal antibodies, such as HP2/1, HP2/4, L25 and P4C2, are described, e.g., in U.S. Pat. No. 6,602,503; Sanchez-Madrid et al, (1986) Eur. J. Immunol 16:1343-1349; Hemler et al, (1987) J Biol. Chem. 2:11478-11485; Issekutz et al. (1991) J Immunol 147: 109 (TA-2 mab); Pulido et al. (1991) J Biol. Chem. 266: 10241-10245; and U.S. Pat. No. 5,888,507). The contents of the aforesaid publications (including the antibody compositions, dosages, methods of administration and production) are incorporated herein by reference in their entirety.

### Dimethyl Fumarate (Tecfidera®)

Dimethyl fumarate, DMF, (Tecfidera®) is a fumaric acid ester. DMF is thought to decrease leukocyte passage through the blood brain barrier and exert neuroprotective effects by the activation of antioxidative pathways, specifically through activation of the Nrf-2 pathway (Lee et al. (2008) Int MS Journal 15: 12-18). Research also suggests that BG-12® has the potential to reduce the activity and impact of inflammatory cells on the CNS and induce direct cytoprotective responses in CNS cells. These effects may enhance the CNS cells' ability to mitigate the toxic inflammatory and oxidative stress that plays a role in MS pathophysiology.

### Glatiramer Acetate (Copaxone®)

Copaxone® (glatiramer acetate) consists of the acetate salts of synthetic polypeptides, specifically the four naturally occurring amino acids: L-glutamic acid, L-alanine, L-tyrosine, and L-lysine (Bornstein et al. (1987) N Engl J Med. 317: 408-414). Copaxone® exhibits structural similarity to myelin basic protein and is thought to function as an immune modulator by shifting the T helper cell type 1 response towards a T helper cell type 2 response (Duda et al. (2000) J Clin Invest 105: 967-976; Nicholas et al. (2011) Drug Design, Development, and Therapy 5: 255-274).

### Mitoxantrone (Novantrone®, an Anthracenedione Molecule)

Mitoxantrone is an anthracenedione molecule (1,4-dihydroxy-5,8-bis[2-(2-hydroxyethylamino) ethylamino]-anthracene-9,10-dione) and a type II topoisomerase inhibitor that disrupts DNA synthesis and repair of cells. It is used to treat cancers and MS. Mitoxantrone is used to treat several forms of advancing MS, including secondary progressive MS, progressive relapsing MS, and advanced relapsing-remitting MS.

For example, mitoxantrone is effective in slowing the progression of secondary progressive MS and extending the time between relapses in relapsing-remitting MS and progressive relapsing MS (Fox E (2006) Clin Ther 28 (4): 461-74).

### Fingolimod (Gilenya®; Sphingosine 1-Phosphate Receptor Modulator)

Fingolimod is an immunomodulating drug, approved for treating MS. It has reduced the rate of relapses in relapsing-remitting multiple sclerosis by over half, but may have serious adverse effects. Fingolimod is a sphingosine 1-phosphate receptor modulator, which sequesters lymphocytes in lymph nodes, preventing them from moving to the central nervous system for autoimmune responses in MS.

### Antibodies to the Alpha Subunit of the IL-2 Receptor of T Cells (CD25) (e.g., Daclizumab HYP; ZINBRYTA®)

An antibody to the alpha subunit of the IL-2 receptor of T cells (CD25), e.g., daclizumab HYP, can be used in the methods and compositions disclosed herein. Daclizumab HYP is a therapeutic humanized monoclonal antibody to the alpha subunit of the IL-2 receptor of T cells (CD25). Daclizumab HYP showed efficacy in reducing lesions and annualized relapse rate in patients with relapsing-remitting multiple sclerosis (Kappos et al. (2015). N Engl. J. Med. 373 (15): 1418-28).

### Antibody Against CD52, e.g., Alemtuzumab

Antibodies against CD52, e.g., alemtuzumab (currently under further development as Lemtrada®), bind to CD52, which is a protein present on the surface of mature lymphocytes, but not on stem cells. Phase III studies reported positive results comparing alemtuzumab with Rebif® (high-dose subcutaneous interferon beta-1a) in the treatment of patients with relapsing-remitting MS (RRMS). Alemtuzumab has been approved in Europe.

### Antibody to CD20, e.g., Ocrelizumab

Antibodies against CD20, e.g., ocrelizumab, rituximab, ofatumumab, target mature B lymphocytes. Phase 2 clinical studies of rituximab and ocrelizumab in relapse remitting MS have demonstrated a statistically significant reduction in disease activity measured by brain lesions (e.g., measured by MRI scans) and relapse rate compared to placebo. Phase 3 studies of ocrelizumab showed both reduction in relapse rate and disability compared to interferon beta-1a (e.g., Rebif®).

### Inhibitors of Dihydroorotate Dehydrogenase, e.g., Teriflunomide

Inhibitors of dihydroorotate dehydrogenase, e.g., teriflunomide, inhibit pyrimidine synthesis. Teriflunomide (also known as A77 1726 or) is an active metabolite of leflunomide. Teriflunomide inhibits rapidly dividing cells, including activated T cells, which are thought to drive the disease process in MS. Teriflunomide was investigated in clinical trials as a medication for treating MS. (Vollmer EMS News (May 28, 2009)).

### Steroids

Steroids, e.g., corticosteroid, and ACTH agents can be used to treat acute relapses in relapsing-remitting MS or secondary progressive MS. Such agents include, but are not limited to, Depo-Medrol®, Solu-Medrol®, Deltasone®, Delta-Cortef®, Medrol®, Decadron®, and Acthar®.

One or more of the aforesaid immunomodulatory agents can be used in combination with the anti-LINGO-1 antibody (or LINGO-1 binding fragment thereof) disclosed herein.

The following are examples of the practice of the invention. They are not to be construed as limiting the scope of the invention in any way.

### EXAMPLES

The anti-LINGO-1 antibody-containing formulation was developed to enable a broad range of parenteral delivery options (subcutaneous (SC, intramuscular (IM) and intravenous (IV)) across a wide clinical dose range. To maximize drug product presentation flexibility, a high concentration formulation was targeted. Success criteria involved achieving a stable product quality profile, while also minimizing product viscosity. The formulation described below is appropriate for parenteral administration (including subcutaneous, intramuscular and intravenous administration) when administered undiluted or when diluted (e.g., into 50 ml or 100 ml) in appropriate intravenous vehicles (0.9% saline or 5% dextrose for infusion).

### Example 1: Early Buffer Component Screen

Formulation development activities were done to investigate high concentration stability. As shown below, these activities identified histidine to be a superior buffer system, compared to the citrate buffer used in early versions of the formulation.

The formulations tested were as follows:

Formulation 1A: 50 mg/ml Li81, 10 mM citrate, 160 mM arginine hydrochloride, and 0.03% polysorbate 80
Formulation 2A: 50 mg/ml Li81, 10 mM histidine (as free-base form histidine, histidine hydrochloride, or a combination of free-base form histidine and histidine hydrochloride), 160 mM arginine hydrochloride, and 0.03% polysorbate 80
Formulation 2E: 50 mg/ml Li81, 10 mM histidine (as free-base form histidine, histidine hydrochloride, or a combination of free-base form histidine and histidine hydrochloride), 160 mM arginine hydrochloride, 10 mM methionine, and 0.03% polysorbate 80
Formulation 3A: 175 mg/ml Li81, 10 mM citrate, 160 mM arginine hydrochloride, and 0.03% polysorbate 80
Formulation 4A: 175 mg/ml Li81, 10 mM histidine (as free-base form histidine, histidine hydrochloride, or a combination of free-base form histidine and histidine hydrochloride), 160 mM arginine hydrochloride, and 0.03% polysorbate 80
Formulation 4E: 175 mg/ml Li81, 10 mM histidine (as free-base form histidine, histidine hydrochloride, or a combination of free-base form histidine and histidine hydrochloride), 160 mM arginine hydrochloride, 10 mM methionine, and 0.03% polysorbate 80

For these studies, Samples were analyzed via size-exclusion chromatography (SEC) using a TSK-gel G3000SWXL column (7.8 mm×30 cm, 5 μm particle size) connected to a Waters HPLC instrument with A280 detector. All dimeric and higher-order soluble aggregate species (referred to as % total aggregate) for each sample were determined using standard integration approaches on Empower 2 software from Waters Corporation (Milford, Mass.).

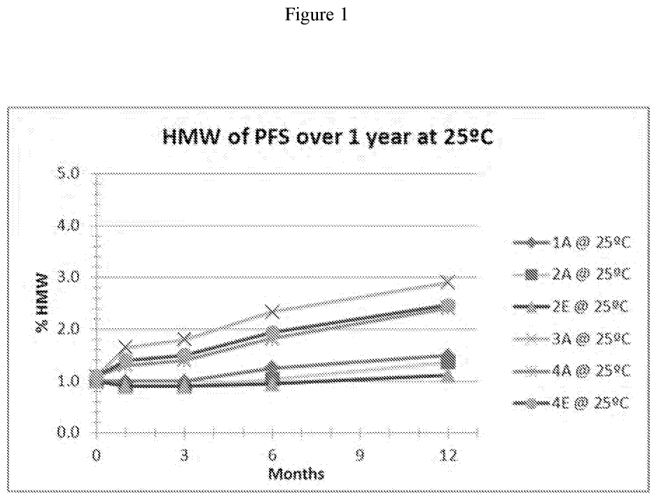

FIG. 1 shows long term stability data of formulations of anti-Lingo varying the buffer species while holding other formulation conditions consistent. As FIG. 1 shows, Formulation 4A (histidine, no citrate) shows better long-term stability than Formulation 3A (citrate, no histidine).

Thus, based on these results, citrate was removed from the formulation and replaced with histidine.

### Example 2: Investigation into Arginine HCl and pH Level

A formulation design of experiment was executed to investigate the relationship of selected anti-Lingo formulation conditions—protein concentration, pH and arginine HCl concentration with regard to their effect on stability and viscosity.

For these studies, the average viscosity was measured over three different shear rates between 480-3900 s⁻¹. The viscosity behavior was confirmed to be Newtonian over the range of shear rates. Viscosity data represent an average of three readings measured at a shear rate between 480-3900 s⁻¹. All viscosity measurements were performed using a m-VROC syringe-based viscometer supplied by RheoSense Inc. (San Ramon, Calif.).

The samples were analyzed via size-exclusion chromatography (SEC) using a TSK-gel G3000SWXL column (7.8 mm×30 cm, 5 μm particle size) connected to a Waters HPLC instrument with A280 detector. All dimeric and higher-order soluble aggregate species (referred to as % total aggregate) for each sample were determined using standard integration approaches on Empower 2 software from Waters Corporation (Milford, Mass.).

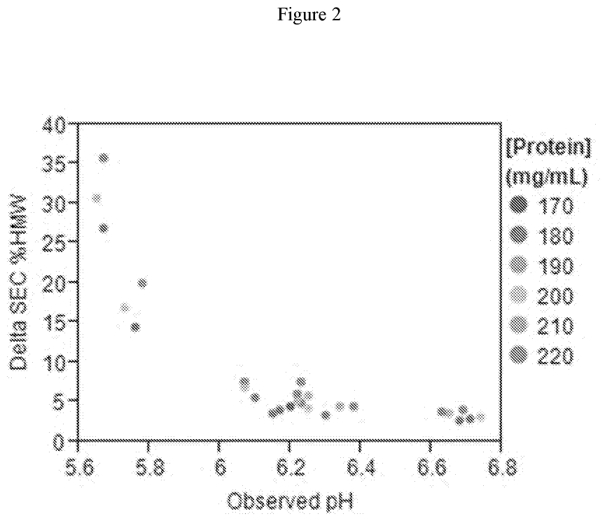

The percentage of high molecular weight (HMW) observed was plotted by pH of the formulation. As FIG. 2 shows, aggregation increases as formulation pH decreases, with concentration dependent aggregation more apparent at lower pH. For example, at pH 6.6-6.8, all concentrations had approximately the same low amount of aggregation. However, at a pH between about 6.05 and 6.3, the highest protein concentration (220 mg/ml) had the highest amount of aggregation while the lowest protein concentration (170 mg/ml) were at the same low level that was seen at the highest pH. At the lowest, pH (e.g., below 5.8), the highest concentration (220 mg/ml) had the highest amount of aggregation while the lowest concentration (170 mg/ml) had the lowest amount of aggregation.

These results suggested that that the optimal formulation pH for anti-Lingo is greater than (>) 6.2.

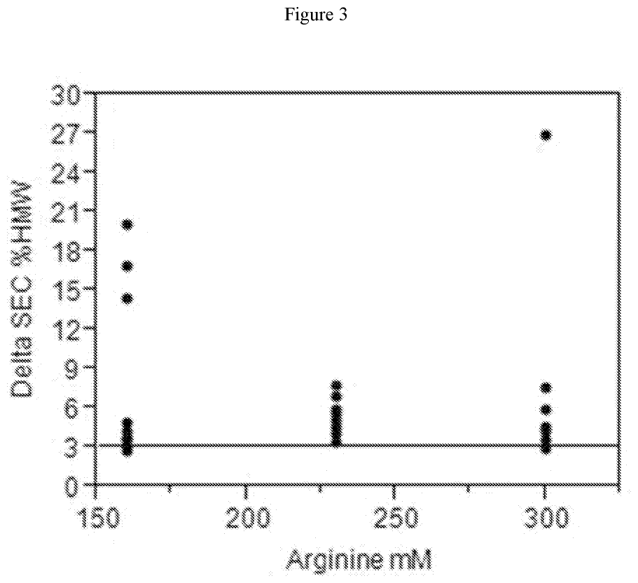

The amount of arginine in the formulation was next analyzed. The results showed that increasing the arginine HCl concentration in the formulation from 160 mM to as much as 300 mM had a negligible impact on stability and solution viscosity (see FIG. 3). In other words, arginine HCl concentration in the formulation does not significantly impact anti-Lingo 1 aggregation.

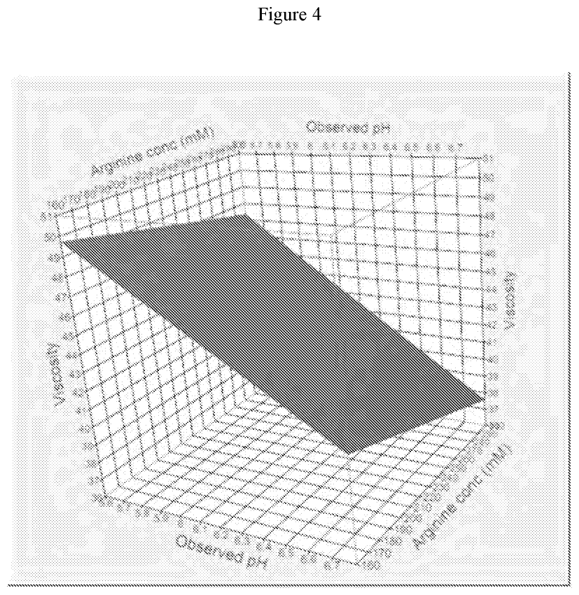

However, when looking at multiple variables (e.g., pH, arginine concentration, and viscosity) it is clear that formulation pH has a significant effect on the viscosity of the anti-Lingo 1 containing formulation (see FIG. 4).

In other words, formulation pH was found to have substantial effect on viscosity (FIG. 4), with viscosity decreasing as formulation pH increases.

Taken together, these results suggest that an optimal formulation pH for an anti-Lingo 1 containing formulation is >6.2. Based on this work, a formulation pH of 6.5 is appropriate for anti-Lingo-1 stability and viscosity.

### Example 3: High Concentration Formulation Excipient Screening

An excipient screening study was performed to identify excipients that can help reduce viscosity while maintaining desired quality attributes. This work focused on identifying excipients that can maintain both stability (as indicated by aggregation by size exclusion chromatography) and a desired viscosity level at a high protein concentration. Excipient levels were designed to be isotonic, to ensure compatibility with parenteral injection routes of administration. The samples were analyzed via size-exclusion chromatography (SEC) using a TSK-gel G3000SWXL column (7.8 mm×30 cm, 5 μm particle size) connected to a Waters HPLC instrument with A280 detector. All dimeric and higher-order soluble aggregate species (referred to as % total aggregate) for each sample were determined using standard integration approaches on Empower 2 software from Waters Corporation (Milford, Mass.).

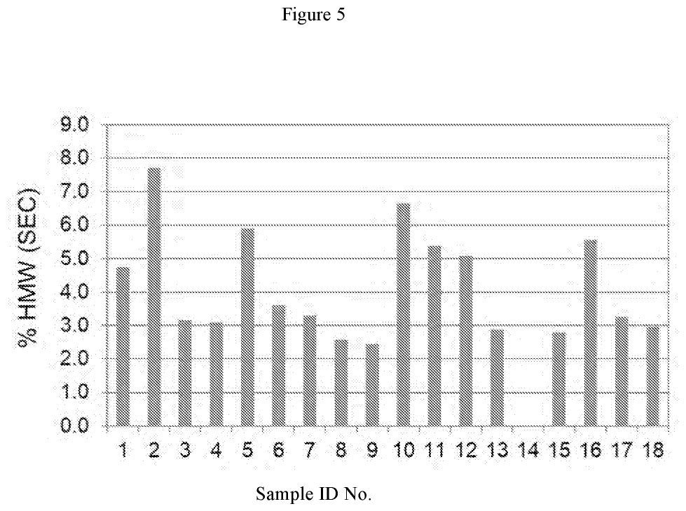

All formulations in this example included anti-Lingo-1 antibody (e.g., Li81) at 225 mg/mL, 20 mM histidine buffer (combination of the free-base form of histidine and histidine hydrochloride) and 0.03% polysorbate 80, at pH 6.5. Table 4 shows the sample ID number and the amount of additional excipient used in FIG. 5.

TABLE 4

| | Excipient in addition to 225 mg/ml protein, |
|---|---|
| ID | 20 mM his/hisHCl, 0.03% polysorbate 80, pH 6.5 |
| 1 | 150 mM NaCl |
| 2 | 150 mM NaCl, 25 mM CaCl2 |
| 3 | 80 mM argHCl (arginine hydrochloride) |
| 4 | 160 mM argHCl |
| 5 | 160 mM argHCl, 25 mM CaCl2 |
| 6 | 160 mM argHCl, 75 mM glutamate |
| 7 | 80 mM argHCl, 75 mM LysHCl |
| 8 | 80 mM argHCl, 150 mM proline |
| 9 | 160 mM argHCl, 25 mM methionine |
| 10 | 25 mM CaCl2 |
| 11 | 75 mM glutamate |
| 12 | 150 mM KCl |
| 13 | 300 mM sucrose |
| 14 | 300 mM trehalose |
| 15 | 300 mM proline |
| 16 | 150 mM LysHCl (lysine hydrochloride) |
| 17 | 300 mM glycine |
| 18 | 300 mM alanine |

Change in high molecular weight species (aggregation) was measured over 3 months of storage at 40° C. for anti-Lingo formulated with various excipients. FIG. 5 provides the stability data of the 18 formulations set forth in Table 2 above. Note that formulations with less than or equal to 3% HMV (SEC) in FIG. 5 (e.g., ID 9 from Table 4) have a desired stability profile while those formulations with greater than 3% HMV (SEC) in FIG. 5 (e.g., ID 2 from Table 4) have a less desirable or undesirable stability profile. As FIG. 5 shows, arginine HCl (ID 3 and 4 from Table 4), proline (ID 15 from Table 4) and sucrose (ID 13 from Table 4) stabilize aggregation of anti-Lingo 1 antibody. The data in FIG. 5 also show that the combination of arginine HCl and methionine (ID 9 from Table 4), and the combination of arginine HCl and proline (ID 8 from Table 4) stabilize anti-Lingo aggregation. A comparison of formulation ID 3 and 4 from Table 4 and in FIG. 5 shows the stability benefit of arginine HCl is present at 80 mM, and further stability is not provided with increased concentrations of arginine HCl.

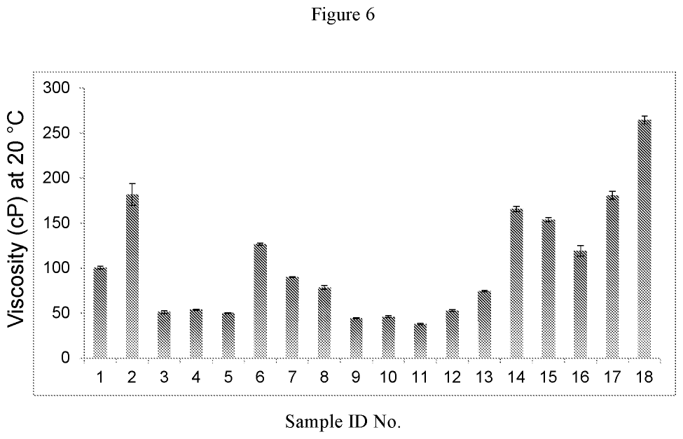

Additional excipients were added to the starting formulation (i.e., anti-Lingo-1 antibody (e.g., Li81) at 225 mg/mL, 20 mM histidine buffer and 0.03% polysorbate 80, at pH 6.5) to create new formulations were made to test viscosity. Table 5 lists these new formulations and their results are presented in FIG. 6.

TABLE 5

| | Excipient in addition to 225 mg/ml protein |
|---|---|
| | (e.g., Li81 antibody), 20 mM histidine buffer (free-base |
| | form histidine, histidine hydrochloride, or a combination |
| | of free-base form histidine and histidine hydrochloride), |
| ID | 0.03% polysorbate 80, pH 6.5 |
| 1 | 25 mM CaCl2 |
| 2 | 75 mM glutamate |
| 3 | 80 mM argHCl (arginine hydrochloride) |
| 4 | 80 mM argHCl, 75 mM LysHCl |
| 5 | 80 mM argHCl, 150 mM proline |
| 6 | 150 mM KCl |
| 7 | 150 mM NaCl |
| 8 | 150 mM NaCl, 25 mM CaCl2 |
| 9 | 160 mM argHCl |
| 10 | 160 mM argHCl, 25 mM CaCl2 |
| 11 | 160 mM argHCl, 25 mM methionine |
| 12 | 160 mM argHCl, 75 mM glutamate |
| 13 | 160 mM LysHCl (lysine hydrochloride) |
| 14 | 300 mM alanine |
| 15 | 300 mM glycine |
| 16 | 300 mM proline |
| 17 | 300 mM sucrose |
| 18 | 300 mM trehalose |

The average viscosity was measured over three different shear rates between 480-3900 s⁻¹. The viscosity behavior was confirmed to be Newtonian over the range of shear rates. Viscosity data represent an average of three readings measured at a shear rate between 480-3900 s⁻¹. All viscosity measurements were performed using a m-VROC syringe-based viscometer supplied by RheoSense Inc. (San Ramon, Calif.).

FIG. 6 provides formulation viscosity measurement data of the formulations set forth in Table 5 above. Note that formulations with less than or equal to 50 cP at 20° C. in FIG. 6 (e.g., ID 11 from Table 5) are amenable to self-administration while those formulations with greater than 50 cP at 20° C. in FIG. 6 (e.g., ID 18 from Table 5) are less amenable or are not amenable to self-administration. As FIG. 6 shows, formulations containing Arginine HCl most effectively reduce viscosity (formulation IDs 3, 4, 5, 9, 10, 11, 12 from Table 5). Evaluation of excipient combinations with and without arginine HCl demonstrates that arginine HCl is the excipient that is driving viscosity reduction in the formulations. A comparison of formulation IDs 3 and 9 from Table 5 and FIG. 6 demonstrate the viscosity reduction benefit of arginine HCl is apparent at 80 mM, and substantial additional viscosity reduction is not achieved by increasing arginine HCl concentration (i.e. to 160 mM).

Evaluation of the stability and viscosity data of this Example revealed arginine HCl is beneficial for stability and useful for viscosity management. Addition of proline or methionine to arginine HCl based formulations demonstrate a stability benefit. The addition of sucrose to an arginine HCL based formulation may offer a stability benefit.

### Example 4: Stability Evaluation

The formulations set forth in Table 6 below were selected for long term stability evaluation.

TABLE 6

| Sample ID | | Viscosity |
|---|---|---|
| No. | Formulation | (cP) |
| 1 | 200 mg/ml anti-Lingo-1, 20 mM histidine (free-base form | 28 |
| | histidine, histidine hydrochloride, or a combination of free-base | |
| | form histidine and histidine hydrochloride), 160 mM arginine | |
| | HCl, 0.05% polysorbate 80, pH 6.5 | |
| 2 | 200 mg/ml anti-Lingo-1, 20 mM histidine (free-base form | 29 |
| | histidine, histidine hydrochloride, or a combination of free-base | |
| | form histidine and histidine hydrochloride), 160 mM arginine | |
| | HCl, 10 mM methionine, 0.05% polysorbate 80, pH 6.5 | |
| 3 | 200 mg/ml anti-Lingo-1, 20 mM histidine (free-base form | 28 |
| | histidine, histidine hydrochloride, or a combination of free-base | |
| | form histidine and histidine hydrochloride), 160 mM arginine | |
| | HCl, 10 mM methionine, 0.05% polysorbate 80, pH 7.0 | |
| 4 | 200 mg/ml anti-Lingo-1, 20 mM histidine (free-base form | 57 |
| | histidine, histidine hydrochloride, or a combination of free-base | |
| | form histidine and histidine hydrochloride), 160 mM arginine | |
| | HCl, 10 mM methionine, 0.05% polysorbate 80, pH 6.5 | |
| 5 | 200 mg/ml anti-Lingo-1, 20 mM histidine (free-base form | 35 |
| | histidine, histidine hydrochloride, or a combination of free-base | |
| | form histidine and histidine hydrochloride), 80 mM arginine | |
| | HCl, 160 mM proline, 0.05% polysorbate 80, pH 6.5 | |
| 6 | 200 mg/ml anti-Lingo-1, 20 mM histidine (free-base form | 41 |
| | histidine, histidine hydrochloride, or a combination of free-base | |
| | form histidine and histidine hydrochloride), 80 mM arginine | |
| | HCl, 10 mM methionine, 5% sucrose, 0.05% polysorbate 80, pH 6.5 | |

As shown above in Table 6, all formulations contained 20 mM histidine/histidine HCl buffer (free-base form histidine, histidine hydrochloride, or a combination of free-base form histidine and histidine hydrochloride) and 0.05% polysorbate 80, and all formulations are arginine HCl based. Arginine HCl in combination with proline and arginine HCl in combination with sucrose were investigated. Slightly elevated pH (7.0) and protein concentration (230 mg/mL) were also investigated.

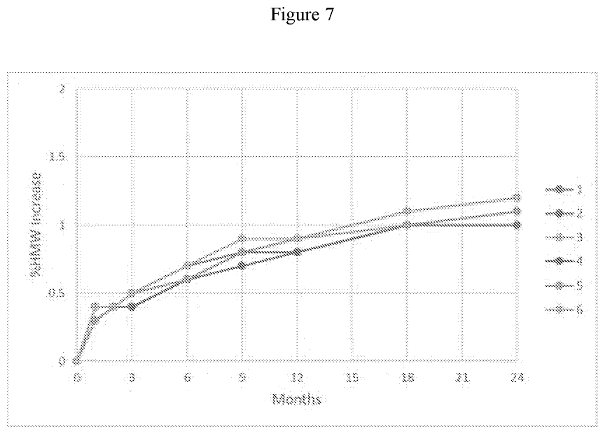

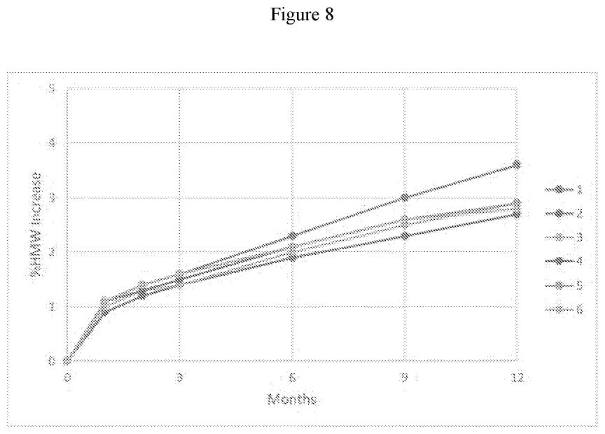

FIGS. 7 and 8 show increase in high molecular weight species (aggregate) of the formulations set forth in Table 6 above over time at 5° C. and 25° C., respectively. As FIGS. 7 and 8 show, a stability benefit was observed when methionine or proline were included in arginine HCl based formulations. Acceptable stability was observed when sucrose is included in the formulation; however, the inclusion of sucrose resulted in an elevated viscosity. Inclusion of proline in the formulation resulted in a reduction in anti-Lingo oxidation, compared to formulation 1, which does not contain methionine (a known antioxidant).

Of the six formulations examined in this Example 4, 200 mg/mL anti-Lingo-1, 20 mM histidine (free-base form histidine, histidine hydrochloride, or a combination of free-base form histidine and histidine hydrochloride), 160 mM arginine HCl, 10 mM methionine, 0.05% polysorbate 80, pH 6.5 (formulation 2 from Table 6 above) provides the best stability and viscosity profile. Formulation 5 (200 mg/mL anti-Lingo, 20 mM histidine (free-base form histidine, histidine hydrochloride, or a combination of free-base form histidine and histidine hydrochloride), 80 mM arginine HCl, 160 mM proline, 0.05% polysorbate 80, pH 6.5) offers an acceptable alternate formulation for anti-Lingo.

Formulation 2 (from Table 6) offers stability across a broad range of anti-Lingo concentrations, at least from 50-230 mg/mL. The level of arginine HCl required for the desired stability and viscosity profile ranges from 80-160 mM, with the amount above 80 mM contributing to an isotonic solution appropriate for parenteral injection. The level of methionine can range from 5 mM to 25 mM and level of polysorbate can range from 0.01-0.09% (w/v).

Similar ranges are appropriate for formulation 5 (Table 6), with proline driving the tonicity above 80 mM arginine HCl. Note methionine is not included in this formulation.

### Example 5: Long Term Stability of Target Formulation

The target formulation of 200 mg/mL anti-Lingo-1, 20 mM histidine (free-base form histidine, histidine hydrochloride, or a combination of free-base form histidine and histidine hydrochloride), 160 mM arginine HCl, 10 mM methionine, 0.05% polysorbate 80, pH 6.5 was manufactured using a representative pilot drug substance process and subsequently filled into glass vials for long term drug product stability. The % high molecular weight (% HMW) was measured by SEC using an ACQUITY UPLC system, Acquity BEH200 SEC guard and analytical column and UV detection. At each timepoint, the sample was diluted to 75 mg/mL in SEC running buffer. 0.3 μL (25 μg) of each sample was injected onto the column and eluted with the running buffer: 100 mM Sodium Phosphate, 200 mM NaCl, pH 6.8 at a flow rate of 0.35 mL/min. The run time for each sample was 10 mins.

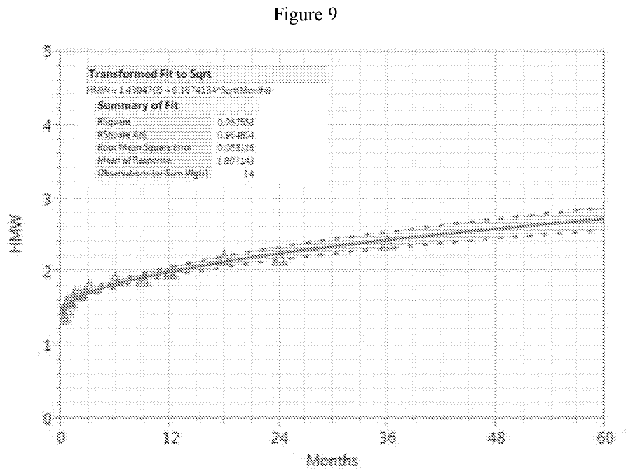

Percent aggregates by size exclusion chromatography data, provided in FIG. 9, demonstrated that 200 mg/mL anti-Lingo-1 in the target formulation is stable for a period of at least 4 years at the intended storage condition of 2-8° C. Aggregation by SEC is the leading indicator of stability.

The formulation, 200 mg/mL anti-Lingo-1 antibody (e.g., Li81), 20 mM histidine (free-base form histidine, histidine hydrochloride, or a combination of free-base form histidine and histidine hydrochloride), 160 mM arginine HCl, 10 mM methionine, 0.05% polysorbate 80, pH 6.5 meets stability and viscosity requirements. Furthermore, the nominated formulation is an isotonic solution, appropriate for parenteral administration. The formulation is amenable to subcutaneous and intramuscular injection, when injected undiluted. The formulation is also appropriate for intravenous infusion, undiluted or diluted to a concentration as low as 1 mg/mL in 0.9% saline (NaCl) or 5% dextrose vehicle.

### Example 6: Long Term Stability of Target Formulation

Table 7 provides a non-limiting recipe for making one of the formulations described herein:

TABLE 7

| Component name | Quantity |
|---|---|
| Li81 antibody | 200 mg |
| L-histidine | 2.2 mg |
| L-arginine hydrochloride | 33.7 mg |
| L-histidine hydrochloride monohydrate | 1.2 mg |
| L-methionine | 1.5 mg |
| Polysorbate 80 | 0.5 mg |
| Sterile water | 0 mg |

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A pharmaceutical composition comprising an anti-LINGO-1 antibody or LINGO-1-binding fragment thereof, histidine, and at least one excipient selected from the group consisting of proline and methionine, wherein the anti-LINGO-1 antibody or LINGO-1-binding fragment comprises an immunoglobulin heavy chain variable domain (VH) and an immunoglobulin light chain variable domain (VL), the VH and VL, respectively, comprising:
(a) VH complementarity determining regions (CDRs), wherein
VH-CDR1 comprises the amino acid sequence set forth in SEQ ID NO:6;
VH-CDR2 comprises the amino acid sequence set forth in SEQ ID NO:7; and
VH-CDR3 comprises the amino acid sequence set forth in SEQ ID NO:8; and
(b) VL CDRs, wherein
VL-CDR1 comprises the amino acid sequence set forth in SEQ ID NO:14;
VL-CDR2 comprises the amino acid sequence set forth in SEQ ID NO:15; and
VL-CDR3 comprises the amino acid sequence set forth in SEQ ID NO:16, and
wherein the composition has a pH of about 6.0 to about 7.0.

2. The pharmaceutical composition of claim 1, wherein the composition further comprises arginine hydrochloride.

3. The pharmaceutical composition of claim 2, wherein the composition comprises arginine hydrochloride at a concentration of about 70 mM to about 170 mM.

4. The pharmaceutical composition of any one of claims 1 to 3, wherein the composition comprises the anti-LINGO-1 antibody or LINGO-1-binding fragment at a concentration of 50 mg/ml to 300 mg/ml.

5. The pharmaceutical composition of any one of claims 1 to 4, wherein the composition comprises the anti-LINGO-1 antibody or LINGO-1-binding fragment at a concentration of 100 mg/ml to 250 mg/ml.

6. The pharmaceutical composition of any one of claims 1 to 5, wherein the composition comprises the anti-LINGO-1 antibody or LINGO-1-binding fragment at a concentration of 150 mg/ml to 225 mg/ml.

7. The pharmaceutical composition of any one of claims 1 to 6, wherein the composition comprises the anti-LINGO-1 antibody or LINGO-1-binding fragment at a concentration of 200 mg/ml.

8. The pharmaceutical composition of any one of claims 1 to 7, wherein the histidine is a combination of free-base form of histidine and histidine hydrochloride.

9. The pharmaceutical composition of any one of claims 1 to 8, wherein the composition comprises histidine at a concentration of about 10 mM to about 30 mM.

10. The pharmaceutical composition of any one of claims 1 to 9, wherein the composition comprises (i) proline at a concentration of about 140 mM to about 180 mM or (ii) methionine at a concentration of about 5 mM to about 15 mM.

11. The pharmaceutical composition of any one of claims 1 to 10, wherein the composition comprises polysorbate-80.

12. The pharmaceutical composition of claim 11, wherein the composition comprises polysorbate-80 at a concentration of 0.01% to 0.1%.

13. The pharmaceutical composition of claim 12, wherein the composition comprises polysorbate-80 at a concentration of 0.03% to 0.08%.

14. The pharmaceutical composition of claim 13, wherein the composition comprises polysorbate-80 at a concentration of 0.05%.

15. The pharmaceutical composition of any one of claims 1 to 14, wherein the composition does not contain citrate.

16. The pharmaceutical composition of any one of claims 1 to 15, wherein the composition has a pH of 6.2 to 6.8.

17. The pharmaceutical composition of any one of claims 1 to 16, wherein the composition has a pH of 6.3 to 6.8.

18. The pharmaceutical composition of any one of claims 1 to 17, wherein the composition has a pH of 6.5.

19. The pharmaceutical composition of claim 1, comprising:
the anti-LINGO-1 antibody or LINGO-1-binding fragment at a concentration of 175 mg/ml to 225 mg/ml;
arginine hydrochloride at a concentration of about 150 mM to about 175 mM;
histidine at a concentration of about 10 mM to about 30 mM;
methionine at a concentration of about 5 mM to about 15 mM; and
polysorbate-80 at a concentration of about 0.01% to about 0.1%,
wherein the composition has a pH of 6.2 to 6.8.

20. The pharmaceutical composition of claim 1, comprising:
the anti-LINGO-1 antibody or LINGO-1-binding fragment at a concentration of 175 mg/ml to 225 mg/ml;
arginine hydrochloride at a concentration of about 70 mM to about 90 mM;
histidine at a concentration of about 10 mM to about 30 mM;
proline at a concentration of about 140 mM to about 180 mM; and
polysorbate-80 at a concentration of about 0.01% to about 0.1%,
wherein the composition has a pH of 6.2 to 6.8.

21. The pharmaceutical composition of claim 1, comprising:
the anti-LINGO-1 antibody or LINGO-1-binding fragment at a concentration of 200 mg/ml;
arginine hydrochloride at a concentration of about 160 mM;
histidine at a concentration of about 20 mM;
methionine at a concentration of about 10 mM; and
polysorbate-80 at a concentration of about 0.05%,
wherein the composition has a pH of 6.5.

22. The pharmaceutical composition of claim 1, comprising:
the anti-LINGO-1 antibody or LINGO-1-binding fragment at a concentration of 200 mg/ml;
arginine hydrochloride at a concentration of about 80 mM;
histidine at a concentration of about 20 mM;
proline at a concentration of about 160 mM; and
polysorbate-80 at a concentration of 0.05%,
wherein the composition has a pH of 6.5.

23. The pharmaceutical composition of any one of claims 1 to 22, wherein the VH comprises a sequence at least 80% identical to SEQ ID NO:5 and the VL comprises a sequence at least 80% identical to SEQ ID NO:13.

24. The pharmaceutical composition of any one of claims 1 to 22, wherein the VH comprises a sequence at least 90% identical to SEQ ID NO:5 and the VL comprises a sequence at least 90% identical to SEQ ID NO:13.

25. The pharmaceutical composition of any one of claims 1 to 22, wherein the VH comprises the amino acid sequence set forth in SEQ ID NO:5 and the VL comprises the amino acid sequence set forth in SEQ ID NO:13.

26. The pharmaceutical composition of any one of claims 1 to 22, wherein the anti-LINGO-1 antibody comprises an immunoglobulin heavy chain and an immunoglobulin light chain, wherein the heavy chain comprises a sequence at least 80% identical to SEQ ID NO:9 and the light chain comprises a sequence at least 80% identical to SEQ ID NO:17.

27. The pharmaceutical composition of any one of claims 1 to 22, wherein the anti-LINGO-1 antibody comprises an immunoglobulin heavy chain and an immunoglobulin light chain, wherein the heavy chain comprises a sequence at least 90% identical to SEQ ID NO:9 and the light chain comprises a sequence at least 90% identical to SEQ ID NO:17.

28. The pharmaceutical composition of any one of claims 1 to 22, wherein the anti-LINGO-1 antibody comprises an immunoglobulin heavy chain and an immunoglobulin light chain, wherein the heavy chain comprises the amino acid sequence set forth in SEQ ID NO:9 and the light chain comprises the amino acid sequence set forth in SEQ ID NO:17.

29. The pharmaceutical composition of any one of claims 1 to 28, comprising a fixed dose of 750 mg of the anti-LINGO-1 antibody or LINGO-1-binding fragment.

30. A method of treating a CNS demyelinating disease in a human subject in need thereof, the method comprising administering to the human subject the pharmaceutical composition of any one of claims 1 to 29.

31. The method of claim 30, wherein the CNS demyelinating disease is multiple sclerosis.

32. The method of claim 30, wherein the human subject is currently, has previously, and/or in the future will be treated with an immunomodulatory agent.

33. The method of claim 32, wherein the immunomodulatory agent is selected from the group consisting of interferon beta 1a, interferon beta 1b, glatiramer acetate, fingolimod, alemtuzumab, cladribine, ocrelizumab, peginterferon beta 1a, dimethyl fumarate, natalizumab, an antibody to the alpha subunit of the interleukin 2 receptor, an inhibitor of dihydroorotate dehydrogenase, a steroid, and a combination of two or more of the forgoing.

34. The method of claim 30, wherein the CNS demyelinating disease is optic neuritis.

35. The method of claim 30, wherein the pharmaceutical composition comprises the anti-LINGO-1 antibody or LINGO-1-binding fragment at a dose of 3 mg per kg, about 5 mg per kg, about 10 mg per kg, about 15 mg per kg, about 30 mg per kg, about 45 mg per kg, about 90 mg per kg, about 100 mg per kg, or about 120 mg per kg of body weight of the human subject.

36. The method of any one of claims 30 to 35, wherein the pharmaceutical composition is administered subcutaneously to the human subject.

37. The method of any one of claims 30 to 35, wherein the pharmaceutical composition is administered intramuscularly to the human subject.

38. The method of any one of claims 30 to 35, wherein the pharmaceutical composition is administered intravenously to the human subject.

39. A syringe comprising the pharmaceutical composition of any one of claims 1 to 29.

40. A kit comprising the syringe of claim 39 and an immunomodulatory agent.

41. The kit of claim 40, wherein the immunomodulatory agent is selected from the group consisting of interferon beta 1a, interferon beta 1b, glatiramer acetate, fingolimod, alemtuzumab, cladribine, ocrelizumab, peginterferon beta 1a, dimethyl fumarate, natalizumab, an antibody to the alpha subunit of the interleukin 2 receptor, an inhibitor of dihydroorotate dehydrogenase, a steroid, and a combination of two or more of the forgoing.

42. A kit comprising one or more syringes comprising the pharmaceutical composition of any one of claims 1 to 29, wherein said one or more syringes is adapted for subcutaneous administration.

43. The kit of claim 42, further comprising instructions for administering the composition subcutaneously.

44. A kit comprising one or more syringes comprising the pharmaceutical composition of any one of claims 1 to 29, wherein said one or more syringes is adapted for intravenous administration.

45. The kit of claim 44, further comprising instructions for administering the composition intravenously.

46. A kit comprising one or more syringes comprising the pharmaceutical composition of any one of claims 1 to 29, wherein said one or more syringes is adapted for intramuscular administration.

47. The kit of claim 46, further comprising instructions for administering the composition intramuscularly.
